(19) United States
(12) Patent Application Publication
(10) Pub. No.: US 2026/0301965 A1
(43) Pub. Date: Oct. 1, 2026

(54) REAL-TIME ADAPTABLE INTERACTIVE AI PERSONA

(71) Applicants: Hussein Ghazy, St Cergue (CH); Sinan Gokce, Geneva (CH)

(72) Inventors: Hussein Ghazy, St Cergue (CH); Sinan Gokce, Geneva (CH)

(21) Appl. No.: 19/416,475

(22) Filed: Dec. 11, 2025

Related U.S. Application Data
(63) Continuation-in-part of application No. 19/096,255, filed on Mar. 31, 2025, now Pat. No. 12,518,461.

(51) Int. Cl. G16H 50/30; G06F 40/30; G06Q 30/04; G06V 40/16; G10L 13/027; G10L 13/033; G10L 17/06; G10L 25/30; G10L 25/63; G10L 25/90

(52) U.S. Cl. CPC G16H 50/30; G06Q 30/04; G10L 17/06; G06F 40/30; G06V 40/176; G10L 13/027; G10L 13/0335; G10L 25/30; G10L 25/63; G10L 25/90; G10L 2025/906

(57) ABSTRACT

An AI Persona Clinician system emulates a supervising Clinician's behavior, tone, facial animation, values, and expertise through a clinician likeness profile. A finite-state controller transitions among normal, caution, and safe-hold states based on multimodal severity scoring (facial emotion, prosody, silence gaps, gaze aversion, keywords) and distribution shift detection from patient baseline.

### CROSS-REFERENCE TO RELATED APPLICATION

This application is a continuation-in-part and incorporates by reference U.S. Non Provisional application Ser. No. 19/096,255 filed on Mar. 31, 2025.

### BACKGROUND OF THE INVENTION

### 1. Field of the Invention

This invention relates to an interactive artificial intelligence system. More particularly, the invention relates to a real-time adaptable interactive AI system that engages in active and reactive discussion with a user.

### 2. Description of the Related Art

Artificial intelligence (AI) conversational agents have evolved significantly over the last decade, yet existing AI systems remain limited in their ability to provide real-time, human-like interactions. Current AI-powered assistants primarily rely on text-based or audio-only exchanges, which lack the multimodal depth necessary for natural engagement. Most AI-driven solutions, such as virtual assistants, chatbots, and customer service applications, fail to synchronize multiple sensory inputs including speech, text, facial expressions, and gaze tracking resulting in rigid, disconnected, and unnatural conversations.

One of the key challenges in AI-human interaction is latency, which refers to the delay between user input and AI response generation. Most AI assistants experience significant delays in generating responses, disrupting the flow of conversation. This latency issue arises due to inefficient natural language processing (NLP) models, lack of predictive response caching, and high computational loads in real-time AI inference. As a result, users often experience unnatural pauses in conversations, leading to reduced engagement and frustration.

Another limitation of conventional AI systems is their inability to recognize and adapt to human emotions in real-time. Current AI assistants operate on rule-based responses or predefined sentiment analysis models that do not dynamically adjust their speech tone, facial expressions, or gestures based on the user's emotional state. Additionally, micro-expression detection and gaze tracking which are essential components of natural human interaction are absent in most AI-driven persona models. This deficiency makes existing AI assistants incapable of expressing empathy, recognizing user stress levels, or modifying interaction intensity dynamically.

Furthermore, existing AI systems lack real-time multimodal, contextual awareness and long-term memory, making interactions feel repetitive, disjointed, and unnatural user interaction. Users are required to repeat contextual details across multiple interactions because the AI system does not retain past engagement data. Without predictive learning capabilities, AI assistants fail to personalize conversations over time, limiting their usefulness in therapy, education, professional mentorship, and long-term engagement scenarios.

Currently, there are no useful alternatives that effectively assist a user in real-time, latency-optimized, context-and-emotion-aware, and adaptive AI-human interactions. Accordingly, there is a need for a method and system to overcome the disadvantages of current AI systems.

### SUMMARY OF THE INVENTION

The present disclosure provides a real-time adaptable interactive AI persona.

The foregoing and/or other features and utilities of the present disclosure may be achieved by providing a method, including converting an audio input to a text input, generating, by an artificial intelligence (AI) persona, at least one of a visual output and an audio output based on the text input and a face input, synchronizing, by the AI persona, at least one of the visual output and the audio output to movement of a rendered facial image, and modifying, by the AI persona, at least one of the visual output and the audio output in real-time in response to real-time changes in the audio input and the face input.

The generating at least one of a visual output and an audio output includes generating at least one of the visual output and the audio output based on a predicted response cached by the AI persona.

The predicted response of the AI persona uses at least one context-aware vector representation of at least one predicted query.

The method further includes generating a plurality of responses corresponding to the at least one predicted query, wherein each of the plurality of responses is unique and modifies at least one of the visual output and the audio output, and modifying each of the plurality of responses based on the text input and the face input, and adjusting engagement style based on recurring sentiment trends and detected behavioral patterns.

The modifying at least one of the visual output and audio output includes modifying at least one of the visual output and audio output based on a plurality of multimodal inputs and reinforcement learning-based adaptation for AI persona refinement over time.

Each of the plurality of multimodal inputs is at least one of voice tone, facial expression, and content of the text input, and the AI persona continuously updates its response model using reinforcement learning and past user interactions.

The face input is at least one of a facial expression, a gaze, and facial micro-expressions for stress detection and engagement tracking.

The audio input is at least one of tone, pitch, and speed.

The foregoing and/or other features and utilities of the present disclosure may also be achieved by providing an artificial intelligence (AI) persona system, including one or more processing units, the one or processing units configured to: convert an audio input to a text input, generate at least one of a visual output and an audio output based on the text input and a face input, synchronize at least one of the visual output and the audio output to movement of a rendered facial image, and modify at least one of the visual output and the audio output in real-time in response to real-time changes in the audio input and the face input.

The one or more processing units are configured to generate at least one of the visual output and the audio output based on a predicted response that is cached.

The one or more processing units are configured to determine the predicted response using at least one context-aware vector representation of at least one predicted query.

The one or more processing units are further configured to: generate a plurality of responses corresponding to the at least one predicted query, wherein each of the plurality of responses is unique and modifies at least one of the visual output and the audio output, and modify each of the plurality of responses based on the text input and the face input and adjust engagement style based on recurring sentiment trends and detected behavioral patterns.

The one or more processing units are configured to modify at least one of the visual output and audio output based on a plurality of multimodal inputs and reinforcement learning-based adaptation for AI persona refinement over time.

Each of the plurality of multimodal inputs is at least one of voice tone, facial expression, and content of the text input, and the AI persona continuously updates its response model using reinforcement learning and past user interactions.

The face input is at least one of a facial expression, a gaze, and facial micro-expressions for stress detection and engagement tracking.

The audio input is at least one of tone, pitch, and speed.

The foregoing and/or other features and utilities of the present disclosure may also be achieved by providing a method, including selecting, by an artificial intelligence (AI) persona, at least one of a visual output and an audio output in response to a plurality of inputs based on at least one predicted query stored in a responses database, modifying, by the AI persona, at least one of the visual output and the audio output in response to changes in at least one of the plurality of inputs, while continuously refining AI persona characteristics based on recurring user interaction history, and updating, by the AI persona, the responses database in response to selection of at least one of the visual output and the audio output, ensuring persona adaptation based on evolving user preferences and contextual learning.

Selecting at least one of the visual output and the audio output includes selecting precomputed responses in the responses database based on Boolean match of the at least one predicted query and interaction history, and generating a new response in response to a failed match of the at least one predicted query and updating the responses database with the new response.

The plurality of inputs includes tracking, by the AI persona, a face of a user to detect expression, tracking, by the AI persona, gaze direction to detect eye focus, and transcribing, by the AI persona, audio input to text.

The method further includes integrating, by the AI persona, a hidden watermark into at least one of the visual output and the audio output based on steganographic encoding.

Before explaining the various embodiments of the invention in detail, it is to be understood that the invention is not limited in its application to the details of construction and to the arrangements of the components set forth in the following description or illustrated in the drawings. Rather, the invention is capable of other embodiments and of being practiced and carried out in various ways. Also, it is to be understood that the terminology employed herein is for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

For a better understanding of the invention, its operating advantages and the specific objects attained by its uses, reference should be made to the accompanying drawings and descriptive matter in which there are illustrated preferred embodiments of the invention.

Various objects, features, aspects and advantages of the present embodiment will become more apparent from the following detailed description of embodiments of the embodiment, along with the accompanying drawings in which like numerals represent like components.

### BRIEF DESCRIPTION OF THE DRAWINGS

The present disclosure may be better understood, and its numerous features and advantages made apparent and readily appreciated to those skilled in the art, by referencing the accompanying drawings.

FIG. 1 is a block diagram of an artificial intelligence (AI) system that generates an AI persona that changes based on a plurality of inputs in accordance with some embodiments.

FIG. 2 is a block diagram illustrating components used by an AI persona to generate a response based on a plurality of inputs and adapt the AI persona to the plurality of inputs in accordance with some embodiments.

FIG. 3 is a block diagram of a context-and-emotion aware audio generator (CEAAG) that uses deep learning models and facial recognition to generate speech based on emotion in accordance with some embodiments.

FIG. 4 is a block diagram of a conversation anticipation engine (CAE) that converts predicted queries into embedded models and a response variant generator (RVG) to generate multiple responses for each predicted query in accordance with some embodiments.

FIG. 5 is a block diagram of an input recognition and comparison (IRC) module to compare a query to a preexisting stored query in a database using vector similarity matching and retrieve a precomputed response for a matched query in accordance with some embodiments.

FIG. 6 is a flow diagram illustrating a method for generating the AI persona that changes based on a plurality of inputs in accordance with some embodiments.

FIG. 7 is a flow diagram illustrating a method for generating responses of an AI persona based on matching a query and updating responses by learning facial expressions and emotions in accordance with some embodiments.

FIG. 8 is a flow diagram illustrating a method for generating an AI persona by analyzing facial expressions and identifying emotions in accordance with some embodiments.

FIG. 9 is a block diagram of an AI system that modifies inputs to an AI persona in accordance with some embodiments.

FIG. 10 is a block diagram of a finite state machine supervisory controller (FSMSC) of an AI persona in accordance with some embodiments.

FIG. 11 is a block diagram of an audit log of the FSMSC in accordance with some embodiments.

FIG. 12 is a block diagram of an event recording stage in accordance with some embodiments.

FIG. 13 is a block diagram of a clinical documentation stage in accordance with some embodiments.

FIG. 14 is a block diagram of a cryptographic sealing stage in accordance with some embodiments.

FIG. 15 is a block diagram of an electronic medical record (EMR) integration stage in accordance with some embodiments.

The same elements or parts throughout the figures of the drawings are designated by the same reference characters.

### DETAILED DESCRIPTION OF THE INVENTION

The embodiment and various embodiments can now be better understood by turning to the following detailed description of the embodiments, which are presented as illustrated examples of the embodiment defined in the claims. It is expressly understood that the embodiment as defined by the claims may be broader than the illustrated embodiments described below. Many alterations and modifications may be made by those having ordinary skill in the art without departing from the spirit and scope of the embodiments.

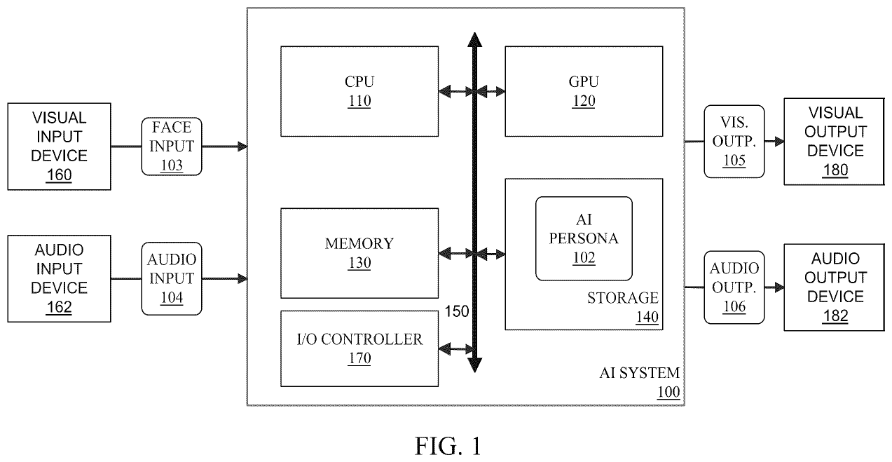

FIG. 1 illustrates a block diagram of an artificial intelligence (AI) system 100 that generates an AI persona 102 that changes based on a plurality of inputs in accordance with some embodiments. The AI system 100 is a system that generally receives different types of inputs for generating the AI persona 102 that interacts and responds to user engagement. For example, the AI persona 102 will generate a visual and an audio output in response to a query from a user. In some embodiments, the AI persona 102 is a program or computer software. However, in different embodiments, the AI persona 102 is a specialized hardware, hardcoded circuit, or hardcoded processor. Alternatively, the AI persona 102 is combination of software and hardware. Accordingly, in different embodiments, the AI system 100 includes a camera, a desktop computer, a laptop computer, a smartphone, a tablet, a microphone, a keyboard, a game console, and the like.

In some embodiments, the AI system 100 includes a central processing unit (CPU) 110, a graphics processing unit (GPU) 120, a memory unit 130, a storage unit 140, a bus 150, a visual input device 160, an audio input device 162, an input/output (I/O) controller 170, a visual output device 180, and an audio output device 182, but is not limited thereto. The CPU 110 is a processing unit that executes instructions, operations, or both based on a program, such as the AI persona 102. In other embodiments, the CPU 110 is a plurality of CPUs 110, which are not shown in the interest of clarity. That is, in other embodiments, the AI system 100 has the plurality of CPUs 110 to execute instructions and/or operations. Alternatively, and/or in addition thereto, in other embodiments, the CPU 110 includes one or more processor cores that execute instructions concurrently or in parallel.

The GPU 120 is a hardware accelerator. In other words, the GPU 120 is a highly specialized processor for performing intensive graphical instructions, operations, or both. In other embodiments, the GPU 120 is a plurality of GPUs 120, which are not shown in the interest of clarity. That is, in other embodiments, the AI system 100 has the plurality of GPUs 120 to execute instructions and/or operations. Alternatively, and/or in addition thereto, in other embodiments, the GPU 120 includes one or more processor cores that execute instructions concurrently or in parallel. Further, in different embodiments, the GPU 120 includes vector processors, coprocessors, non-scalar processors, highly parallel processors, artificial intelligence (AI) processors, inference engines, machine-learning processors, other multithreaded processing units, scalar processors, serial processors, programmable logic devices (simple programmable logic devices, complex programmable logic devices, field programmable gate arrays (FPGAs)), or any combination thereof. The GPU 120 renders a visual output (e.g., an image, a picture, a frame, a stream of frames, a video, a hologram) in response to one or more graphic instructions from the CPU 110 and based on the program, such as the AI persona 102. For sake of brevity, hereafter, collectively the CPU 110 and the GPU 120 are also referred to as one or more processing units.

The memory unit 130 is a transitory storage component such as, for example, a dynamic random-access memory (DRAM). However, in other embodiments, the memory unit 130 includes other types of memory such as, for example, static random-access memory (SRAM), and the like. The memory unit 130 further includes a memory controller, not shown for clarity. The memory controller facilitates the flow of data between the one or more processing units and the memory unit 130. Additionally, the memory controller manages read and/or write operations within memory while correcting errors (e.g., error correction) for detected errors in the memory unit 130. According to some embodiments, the memory unit 130 includes an external memory disposed external to the CPU 110 and the GPU 120 in the AI system 100. The memory unit 130 stores information or data in memory including results of any executed instructions or operations based on the AI persona 102.

The storage unit 140 may include any non-transitory storage medium. For example, the storage unit 140 includes a hard drive, an optical drive (e.g., compact disc (CD), digital versatile disc (DVD), Blu-Ray disc), non-volatile memory (e.g., read-only memory (ROM), Flash memory), and the like. In some embodiments, the storage unit 140 is disposed within the AI system 100, connected to the AI system 100 via an external port, removably connected to the AI system 100, or a cloud-based storage unit connected via a wireless or hardwired network.

The bus 150 facilitates communication between components within the AI system 100.

The CPU 110 and the GPU 120 are both connected to the bus 150 and therefore communicate with each other, the memory unit 130, as well as other components, via the bus 150.

The visual input device 160 includes any type of device capable of scanning visual data. In some embodiments, the visual input device 160 is a camera, a visual data reader, an iris scanner, a facial recognition unit, and a holographic input unit, but is not limited thereto. The visual input device 160 is disposed within the AI system 100, connected to the AI system 100 via an external port, or removably connected to the AI system 100. In some embodiments, the visual input device 160 detects a face input 103 in response to a trigger, such as movement detection within a predetermined distance (e.g., 5 inches, 10 inches, 2 feet, 5 feet, etc.) and within a predetermined area (e.g., angular orientation with respect to the visual input device 160). Alternatively, and/or in addition thereto, the visual input device 160 scans and detects the face input 103 in response to a command input (e.g., take a picture, take a video, etc.) received by the visual input device 160. After the visual input device 160 detects and scans the face input 103 (e.g., a face of a user), the visual input device 160 sends the face input 103 to the memory 103 via the I/O controller 170 for processing by the one or more processing units. Specifically, the AI persona 102 includes instructions executed by the one or more processing units to generate at least one data output based on the face input 103. It will be appreciated that although the face input 103 is referred to as a singular input, the face input 103 may be a plurality of face inputs 103 such as, for example, a stream of video frames.

The audio input device 162 includes any type of device capable of scanning audio data. In some embodiments, the audio input device 162 is a microphone and a voice recognition unit. The audio input device 162 is disposed within the AI system 100, connected to the AI system 100 via an external port, or removably connected to the AI system 100. In some embodiments, the audio input device 162 detects an audio input 104 in response to a trigger, such as movement detection within a predetermined distance (e.g., 5 inches, 10 inches, 2 feet, 5 feet, etc.) and within a predetermined area (e.g., angular orientation with respect to the audio input device 162). Alternatively, and/or in addition thereto, the audio input device 162 scans and detects the audio input 104 in response to a command input (e.g., record an utterance, record speech, etc.) received by the audio input device 162. After the audio input device 162 detects and scans the audio input 104 (e.g., a voice of a user), the audio input device 162 sends the audio input 104 to the memory 103 via the I/O controller 170 for processing by the one or more processing units. Specifically, the AI persona 102 includes instructions executed by the one or more processing units to generate at least one data output based on the audio input 103. It will be appreciated that although the audio input 104 is referred to as a singular input, the audio input 104 may be a plurality of audio inputs 104 such as, for example, a stream of audio.

In some embodiments, the AI system 100 includes the input/output (I/O) controller 170 that includes circuitry to handle input or output operations, as well as other elements of the AI system 100 such as keyboards, mice, printers, external disks, and the like. The I/O controller 170 is connected to the bus 150 so that the I/O controller 170 communicates with CPU 110, the GPU 120, the memory unit 130, and the storage unit 140.

Furthermore, additional types of input devices may be connected to the AI system 100, but are not shown for clarity. For example, additional input devices include an input keyboard, a touchpad, a mouse, a trackball, a stylus, a wireless device reader, and a fingerprint reader.

Based on instructions from the AI persona 102, the one or more processing units execute instructions to generate a persona. More specifically, the AI persona 102 is a deep learning facial animation model that mimics human facial expressions, human behaviors, exhibits emotional awareness, emotion detection, and predicts responses to a query (e.g., a text input, a visual/video/face input, an audio/voice input) from the user in conversation. To exhibit natural reaction and human likeness, the AI persona 102 evaluates a plurality of inputs. For example, the AI persona 102 receives the face input 103 and the audio input 104 and generates a response (e.g., a visual output and an audio output) based on the face input 103 and the audio input 104.

Furthermore, the AI persona 102 may be customized and trained by employing other data formats to ensure an adaptive and contextually rich experience with the AI persona 102. In addition to the face input 103 and the audio input 104, the user may submit a direct text input, such as, for example, from an input keyboard or a stylus (e.g., text input from hand movement). Other data formats include at least one website, documents (e.g., PDF, TXT, PPT, XML, HTML, and the like), and images (e.g., PNG, JPG, TIFF, GIF, SVG, BMP, and the like). Each of the data formats are received by the AI persona 102 for data ingestion and adaptation to the user for the AI persona 102. Moreover, each data format may include different types of content to construct the

AI persona 102. To illustrate, the text used for customization may include articles, transcripts, and/or notes. The audio used for customization may include voice recordings and/or podcasts. The video used for customization may include speech-driven media and/or recorded interviews. The websites used for customization may include eBooks and/or scanned notes. Finally, the images used for customization may include handwritten notes and/or scanned documents.

The AI persona 102 processes the data formats by extracting any information contained therein and used by the AI persona 102 to construct and personalize the rendered persona for the user. The AI persona 102 includes a speech-to-text engine to translate each portion of the audio input 104 into a text query. That is, the audio input 104 is translated to text for additional processing (i.e., entering text-based commands) by the AI persona 102. Alternatively, and/or in addition thereto, the AI persona 102 receives a text query using a direct text input. It is important to note that the AI persona 102 does not need to perform speech conversion for the direct text input. Also, the AI persona 102 includes optical character recognition (OCR) to extract text from images and/or documents (e.g., PDF) that cannot easily extract text. Websites are parsed to retrieve meaningful information. In response to generating the persona, the AI persona 102 embeds content extracted from each data format into a context-aware vector that is stored in the embedded database within the storage unit 140. By employing a vector format to store data, the AI persona 102 generates an index within the embedded database that facilitates rapid retrieval and context awareness. For example, history books, history-focused websites, and/or scanned historical images are used to customize the AI persona 102 into an AI historian persona that retrieves accurate information from the different data formats.

In response to receiving the text query, the AI persona 102 evaluates the text query using an input recognition and comparison (IRC) module. Specifically, the IRC module checks whether the text query has been input (i.e., requested) before, and therefore, is categorized as a preexisting query, or is instead a new query. For a preexisting query (PQ), the IRC module confirms presence of the PQ within an embedded database generated by the AI persona 102 and stored within the storage unit 140. A response lookup module retrieves a preexisting response (a.k.a. a cached response) from a preexisting response (PR) database generated by the AI persona 102 within the storage unit 140 for near-instantaneous delivery of a response. It is important to note that hereinafter, the embedded database and the PR database are described as part of the storage unit 140. However, in different embodiments, the embedded database and the PR database may be in separate, individual storage units (e.g., two storage units) or even stored across a plurality of storage units. In this manner, the cached response substantially reduces latency and provides a faster response for engagement with the user. Hereafter, use of the term, module, refers to a program or computer software for clarity. However, in different embodiments, a module is a specialized hardware, hardcoded circuit, or hardcoded processor. Alternatively, a module is implemented as a combination of software and hardware.

Alternatively, the AI persona 102 employs a text generation (TG) module that includes a large language model (LLM) trained using large amounts of text and data to facilitate text generation. Moreover, in response to the IRC detecting a new query, the TG module generates a new response in response to the IRC module confirming absence of the PQ within the embedded database. Stated differently, the absence of the PQ indicates the new query has never been presented to the AI persona 102 and the AI persona 102 must generate a response relevant and responsive to the new query. Also, the AI persona 102 stores the new query in the embedded database. The TG module transmits the new response or a generated response to a response variant generator (RVG). The RVG includes an LLM trained using large amounts of text and data to facilitate text generation. In particular, the RVG generates a plurality of responses for the new query. As such, the AI persona 102 presents at least one of the plurality of responses based on the new query, as well as additional context. For example, each of the plurality of responses is based on additional factors, such as context of the query, tone of voice in the audio input 104, facial expressions detected in the face input 103, and the like. The RVG stores the plurality of responses in the PR database.

While the TG module generates the new response, a conversation anticipation engine (CAE) operates in parallel. Hereafter, use of the term, engine, refers to a program or computer software for clarity. However, in different embodiments, an engine is a specialized hardware, hardcoded circuit, or hardcoded processor. Alternatively, an engine is implemented as a combination of software and hardware. The CAE analyzes the text input and continued conversation history (e.g., additional utterance and input or query from the user) to predict future queries (i.e., possible queries) and responses. To expedite responsiveness, the AI persona 102 employs the CAE to precompute queries and responses to quickly offer cached responses based on precomputed queries from further inputs by the user and reduce latency.

As described above, the AI persona 102 either retrieves the cached response from the PR database or the AI persona 102 must generate a new response based on a new query that has not been encountered before. In both situations, the cached response or the new response is sent to a context-and-emotion aware audio generation (CEAAG) module. The CEAAG module receives the face input 103 and the audio input 104 in addition to the responses. The CEAAG module generates an audio output 106. The CEAAG module modifies the audio output 106 employing speech synthesis to reflect the emotional state of the user including parameters, such as pitch, speed, cadence, and tone. Further, the CEAAG module adjusts engagement style based on recurring sentiment trends and detected behavioral patterns of the user. In this manner, the audio output 106 will exhibit empathy for the user during conversation. Furthermore, the AI persona 102 employs an emotion aware animated persona generation (EAAPG) module that generates an animated model (e.g., a persona) and outputs a visual output 105. The EAAPG module synchronizes facial expressions, lip movements, stress levels, and micro-expressions (e.g., mood shifts). The EAAPG module responds in real-time to exhibit a natural and human-like persona. It will be appreciated that although the audio output 106 and the visual output 105 are referred to as a singular output, the visual output 105 and the audio output 106 may be a plurality of visual outputs 105 and a plurality of audio outputs 106 such as, for example, a stream of video frames and a stream of audio, respectively.

The visual output device 180 includes any type of device capable of outputting visual data. The visual output device 180 may include a plasma screen, an LCD screen, a light emitting diode (LED) screen, an organic LED screen, a computer monitor, a hologram output unit, a sound outputting unit, or any other type of device that visually or aurally displays data. The visual output device 180 is disposed within the AI system 100, connected to the AI system 100 via an external port, or removably connected to the AI system 100. The visual output device 180 displays the visual output 105 thereon.

The audio output device 182 includes any type of device capable of outputting audio data. The audio output device 182 may include a speaker and any other type of sound outputting unit. The audio output device 182 is disposed within the AI system 100, connected to the AI system 100 via an external port, or removably connected to the AI system 100. The audio output device 182 emits the audio output 106 therefrom.

In some embodiments, the AI persona 102 offers refinement of persona characteristics that adjusts over time and reinforces certain responses based on learning over user behavior. For example, the AI persona 102 has selectable communication styles, such as formal or conversational. The AI persona 102 has different tones for responding, such as empathetic or authoritative. It will be appreciated that despite user customization, the AI persona 102 will modify the visual output 105 and the audio output 106 (e.g., facial expressions, communication style, and tone) as needed based on interaction with the user. The modification of communication by the AI persona 102 may include extracting different predicted responses already stored in the storage unit 140 or generating a new response depending on the query input from the user. For example, the user may select the AI persona 102 to operate as an AI therapist and use a tone that is calming and empathetic during communication. However, the AI persona 102 will modify conversation features (e.g., the tone and facial features) of the visual output 105 and the audio output 106 via a feedback loop in response to detected changes in context from the face input 103 (e.g., frowning, smiling, changing eye gaze, etc.) and/or the audio input 104 (e.g., change in speech pattern, slower speech, increased volume, etc.). The feedback loop is real-time adaptation by the AI persona 102 to continuous input changes from the user and adjust the persona accordingly. Moreover, the AI persona 102 refines conversation engagement through learning from past interactions to ensure future conversations are personalized to the user and demonstrate emotional alignment to the user. That is, the AI persona 102 uses reinforcement learning-based adaptation for the AI persona refinement over time.

In other embodiments, the AI persona 102 processes the plurality of inputs and generates responses using encryption and secure storage protocols. To prevent tampering and ensure secure interactions with the user, the AI persona 102 employs invisible watermarks applied to generated responses and encryption signatures to maintain authenticity and prevent unauthorized use. The watermarks may be applied to the audio output and the visual output using steganographic encoding to prevent unauthorized replication and to verify AI-generated integrity. The watermarks identify responses that have not been modified by an external entity. The AI persona 102 includes a public key verification process that allows authorized users and third parties to verify the generated response is authentic. Moreover, the AI persona 102 has a safeguard layer to continuously monitor the generated responses to ensure they align with data protection laws and ethical guidelines, such as, for example HIPAA, CCPA, GDPR, and SOC II Type II. In other embodiments, the AI persona 102 includes a compliance module with bias detection to ensure the generated responses are fair, balanced, and free from discrimination.

In other embodiments, the AI persona 102 continuously monitors conversations to ensure accuracy and adaptability. The AI persona 102 scans generated responses for speech inconsistencies, contextual mismatches, and sentiment misalignment for possible errors. The AI persona 102 adjusts or modifies any of the generated responses in response to detecting the errors. In particular, the AI persona 102 may modify response strategy, language complexity, speech pacing, and engagement style to enhance communication clarity. Also, in some embodiments, the AI persona 102 employs predictive correction models. That is, the AI persona 102 anticipates and resolves potential errors in the generated responses before occurrence. As such, the AI persona 102 mitigates errors, and maintains cohesion and engagement during conversation.

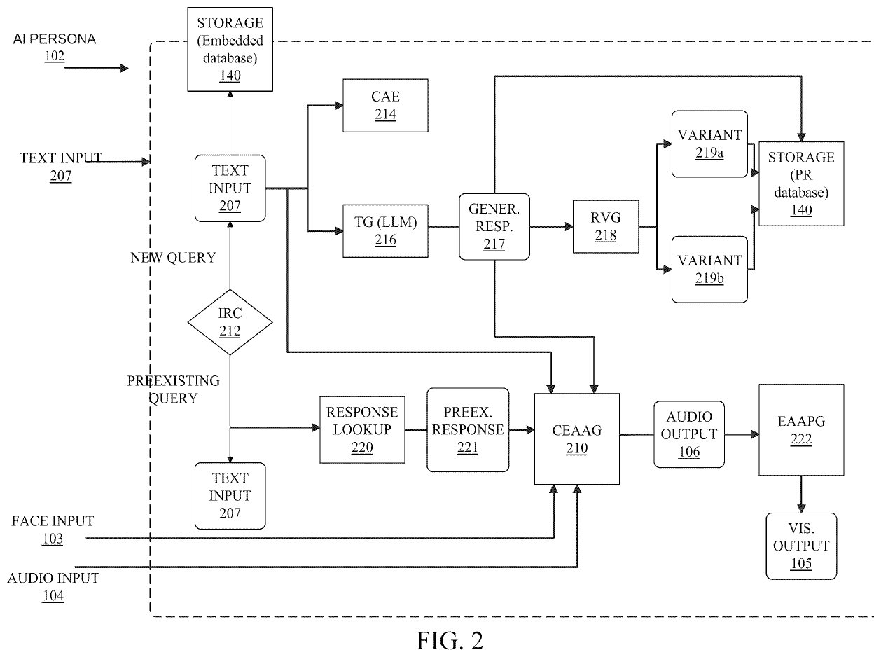

FIG. 2 illustrates a block diagram of components used by an AI persona 102 to generate a response based on a plurality of inputs and adapt the AI persona 102 to the plurality of inputs in accordance with some embodiments. In some embodiments, the AI persona 102 includes a CEAAG module 210, an IRC module 212, a CAE 214, a TG module 216, an RVG 218, a response lookup module 220, and an EAAPG module 222, but is not limited thereto. The AI persona 102 employs the CEAAG module 210 to generate the audio output 106. Specifically, the CEAAG module 210 generates the audio output 106 in response to the AI persona 102 receiving the face input 103, the audio input 104, and a text input 207. The text input 207 may be a direct text input from an input device, such as, for example, from an input keyboard or a stylus. The CEAAG module 210 extracts context from the text query and detects emotional tone from the text input 207, the face input 103, and the audio input 104. The CEAAG module 210 examines spoken words, pitch, and tone from the audio input 104, as well as, facial movements, stress detection, and facial micro-expressions from the face input 103.

To extract context from the text query, the CEAAG module 210 includes AI models. For example, in some embodiments, the CEAAG module 210 includes generative pre-trained transformer models (GPT), bidirectional encoder representations from transformers (BERT), and multimodal transformers. Based on the AI models, the CEAAG module 210 analyzes word relationships to extract context. To analyze speech emotion, the CEAAG module 210 includes deep learning models. For example, in some embodiments, the CEAAG module 210 includes a convolutional neural network (CNN), a Wave2Vec model, and a recurrent neural network (RNN).

Based on the deep learning models, the CEAAG module 210 analyzes speech to recognize speech patterns, vocabulary, and dictation. Finally, the CEAAG module 210 employs facial recognition models. For example, in some embodiments, the CEAAG module 210 extracts facial expressions (e.g., muscle movement for happiness, sadness, frustration), stress level (e.g., sweat on the face, face twitches), facial micro-expressions (e.g., subtle emotional cues), and eye gaze patterns (e.g., indicative of user attention and focus). Also, the CEAAG module 210 analyzes facial landmarks for positioning and orientation, such as eye location, nose location, ear location, mouth location, and the like.

In generating the audio output 106, the CEAAG module 210 combines inputs from the AI models, the deep learning models, and the facial recognition models to generate the audio output 106. The CEAAG module 210 generates the audio output 106 with emotion based on the inputs. To illustrate via an example, the CEAAG module 210 receives the text input 207 stating “I'm having a great day,” the audio input 104 indicates the user has a low tone and indicates the user is actually upset, and the face input 103 indicates the user has a neutral expression. The CEAAG module 210 generates the audio output 106 with empathy having a comforting tone and moderate volume to offer a comforting persona. The AI persona 102 will further modify the response tone of the audio output 106 based on detected emotion from the inputs. The CEAAG module 210 modifies the audio output 106 employing speech synthesis corresponding to the emotional state of the user including parameters, such as pitch, speed, cadence, and tone. Further, the CEAAG module 210 adjusts engagement style based on recurring sentiment trends and detected behavioral patterns of the user. In this manner, the AI persona 102 will exhibit empathy for the user during conversation based on changes in the audio output 106.

Furthermore, the CEAAG module 210 receives the text input 207 from the IRC module 212. The IRC module 212 evaluates the text input 207 based on whether the text input 207 is similar to a prior text input (i.e., has been requested before). The IRC module 212 identifies the text input 207 as a PQ that has been precomputed by the CAE 214 in response to confirming presence of the PQ in the embedded database within the storage unit 140. The response lookup module 220 retrieves a preexisting response (PR) 221 (a.k.a. a cached response 221) from the PR database generated by the AI persona 102 within the storage unit 140 for near-instantaneous delivery of a response. It is important to note that the PR 221 corresponds to the PQ and may be one of a plurality of variants of preexisting responses discussed below. In this manner, the cached response 221 substantially reduces latency and provides a faster response for engagement with the user. Accordingly, the PR response 221 is sent to the CEAAG module 210 to generate the audio output 106 based on the PR response 221.

In the scenario where the IRC module 212 detects the text input 207 as a new query, the AI persona 102 employs the TG module 216 that includes an LLM trained using large amounts of text and data to facilitate text generation. The TG module 216 generates a generated response 217 in response to the IRC module 212 confirming absence of the PQ within the embedded database. Stated differently, the absence of the PQ indicates the new query has never been presented to the AI persona 102 and the AI persona 102 must generate the generated response 217 that is relevant and responsive to the new query. Also, the AI persona 102 stores the new query in the embedded database within the storage unit 140 as well as precomputes additional variant queries based on the new query. It will be appreciated that although the embedded database and the PR database are referred to as being on the same storage unit 140. In different embodiments, the embedded database and the PR database are on separate storage units 140. The TG module 216 transmits the generated response 217 to the RVG 218. The RVG 218 includes an LLM trained using large amounts of text and data to facilitate text generation. In particular, the RVG 218 generates a plurality of variant generated responses 219a, 219b for the new query. FIG. 2 depicts the plurality of variant generated responses as a variant generated response 219a and a variant generated response 219b for clarity. However, in various embodiments, the plurality of variant generated response includes more than two variant generated responses. Each of the plurality of variant generated responses 219a, 219b is responsive to the new query and is based on additional factors, such as context of the text input 207, tone of voice in the audio input 104, facial expressions detected in the face input 103, and the like. As such, the AI persona 102 presents at least one of the plurality of responses based on the new query, as well as additional context. The RVG 218 stores the plurality of variant generated responses 219a, 219b in the PR database.

The CAE 214 operates in parallel with the TG module 216. The CAE 214 analyzes the text input 207 and continued conversation history (e.g., additional utterance and input or query from the user) to predict future queries (i.e., possible queries) and responses. To expedite responsiveness, the AI persona 102 employs the CAE 214 to precompute queries and responses to quickly offer cached responses from further inputs and reduce latency. The CAE 214 stores the precomputed queries in the embedded database and the precomputed responses within the PR database. The precomputed queries are variant queries that anticipate future queries from the user and have corresponding responses that are responsive to those queries.

After generating the audio output 106, the CEAAG module 210 sends the audio output 106 to the EAAPG module 222. The EAAPG module 222 generates an animated model (e.g., a persona) and outputs a visual output 105. The EAAPG module 222 synchronizes facial expressions, lip movements, and micro-expressions (e.g., mood shifts) to the audio output 106. For example, the EAAPG module 222 extracts phoneme sequences and matches them to a facial animation library to ensure precise and accurate lip-sync alignment. Also, animated facial micro-expressions and emotion intensity is generated dynamically to respond to inputs from the user. The EAAPG module 222 responds in real-time to exhibit a natural and human-like persona. Accordingly, the EAAPG module 222 ensures fluid and natural engagement of the AI persona 102 during conversation with the user.

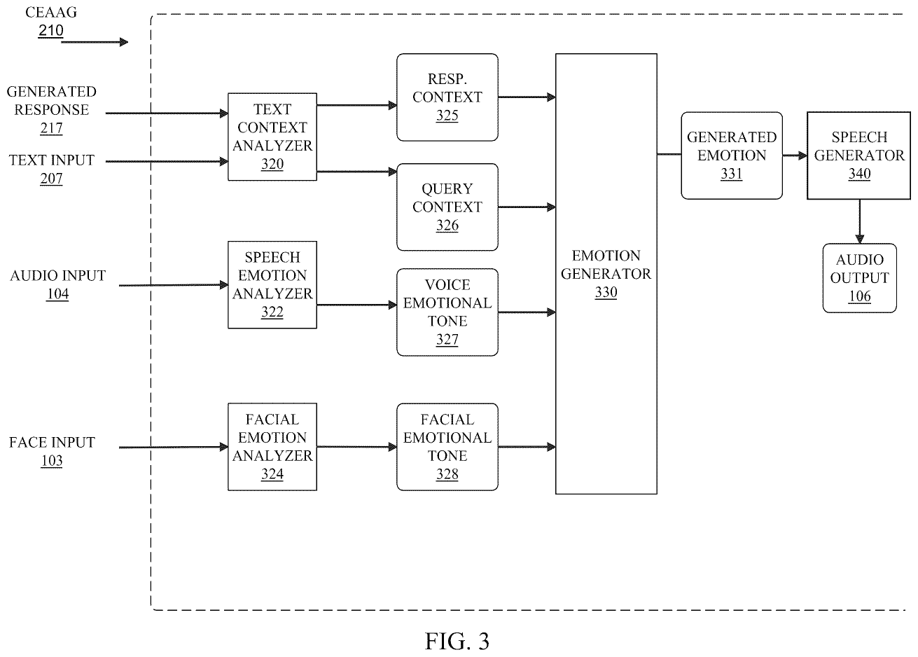

FIG. 3 is a block diagram of a context-and-emotion aware audio generator (CEAAG) module 210 that uses deep learning models and facial recognition to generate speech based on emotion in accordance with some embodiments. To synthesize speech that reflects the emotional state of the user, the CEAAG module 210 employs different analyzers for each type of the plurality of inputs. In some embodiments, the CEAAG module 210 includes a text context analyzer 320, a speech emotion analyzer 322, a facial emotion analyzer 324, an emotion generator 330, and a speech generator 340, but is not limited thereto. As described above, the AI persona 102 employs the CEAAG module 210 to generate the audio output 106. Specifically, the CEAAG module 210 generates the audio output 106 in response to the AI persona 102 receiving the face input 103, the audio input 104, and the text input 207. The text context analyzer 320 receives the text input 207 and analyzes the text input 207 for context, such as order of words and type of words used. To extract context from the text query, the text context analyzer 320 implements at least one AI model. For example, in some embodiments, the text context analyzer 320 includes generative pre-trained transformer models (GPT), bidirectional encoder representations from transformers (BERT), and multimodal transformers. Based on the AI models, the text context analyzer 320 analyzes word relationships to extract context. Furthermore, in some embodiments, the text context analyzer 320 receives the generated response 217 sent from the TG module 216 described above. The text context analyzer 320 analyzes the generated response 217 in the same manner as the text input 207. It will be appreciated that while FIG. 3 depicts the text context analyzer 320 as a single component, in different embodiments, the text input 207 and the generated response 217 are sent to individually separate text context analyzers 320 (e.g., two context analyzers). The text context analyzer 320 sends a response context result 325 to the emotion generator 330 in response to completing analysis of the generated response 217. Similarly, the text context analyzer 320 sends a text context result 326 to the emotion generator 330 in response to completing analysis of the text input 207.

The speech emotion analyzer 322 receives and analyzes the audio input 104 for context, such as words used and manner of speech. To analyze speech emotion, the speech emotion analyzer 322 includes deep learning models. For example, in some embodiments, the speech emotion analyzer 322 includes a convolutional neural network (CNN), a Wave2Vec model, and a recurrent neural network (RNN). Based on the deep learning models, the speech emotion analyzer 322 analyzes speech to recognize speech patterns, vocabulary, and dictation. The speech emotion analyzer 322 sends a voice emotional tone result 327 to the emotion generator 330 in response to completing analysis of the audio input 104.

The facial emotion analyzer 324 receives and analyzes the face input 103 for context, such as facial recognition. To analyze facial emotions and facial expressions, the facial emotion analyzer 324 includes facial recognition models. For example, in some embodiments, the facial emotion analyzer 324 extracts facial expressions, facial micro-expressions, and eye gaze patterns. Based on the facial recognition models, the facial emotion analyzer 324 analyzes the face to recognize face and eye movements. The facial emotion analyzer 324 sends a facial emotional tone result 328 to the emotion generator 330 in response to completing analysis of the face input 103.

The emotion generator 330 employs neural fusion to combine emotion signals from the response context result 325, the text context result 326, the voice emotional tone result 327, and the facial emotional tone result 328. That is, the emotion generator 330 employs neural fusion to combine multiple data sets to facilitate speech generation. As such, the emotion generator 330 modifies emotional tone response based on emotion detected in the results from each analyzer. After combining the emotion signals, the emotion generator 330 generates a generated emotion 331 and send the generated emotion 331 to the speech generator 340. The speech generator 340 employs speech synthesis and AI models to fine tune the speech and generate the audio output 106. In particular, the speech generator 340 modifies at least one of tone, pitch, speed, and resonance to match emotional sentiment of the user. To illustrate via an example, the speech generator 340 may modify the pitch and tone of the audio output 106 prior to generating the audio output 106 in response to the emotion signals indicating an excited user that smiles and/or screams with joy. Based on these signals, the speech generator 340 elevates the pitch and tone to match the emotional sentiment of the user.

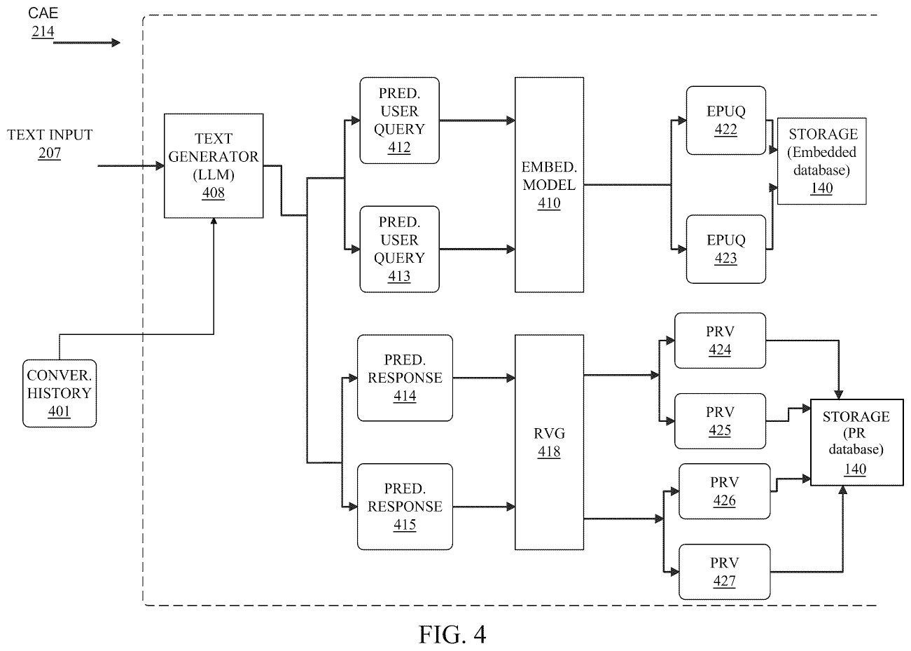

FIG. 4 is a block diagram of a conversation anticipation engine (CAE) 214 that converts predicted queries into embedded models and a response variant generator (RVG) 218 to generate multiple responses for each predicted query in accordance with some embodiments. To expedite responsiveness, the AI persona 102 employs the CAE 214 to precompute queries and responses to quickly offer cached responses from further inputs and reduce latency. In some embodiments, the CAE 214 includes a text generator 408, an embedding model 410, and an RVG 418, but is not limited thereto. The text generator 408 and the RVG 418 operate similar to the TG module 216 and the RVG 218 as described above with respect to FIG. 2. The text generator 408 receives and analyzes the text input 207 and a conversation history 401 (e.g., additional utterance and input or query from the user) to predict and compute future queries (i.e., possible queries) and responses. The text generator 408 generates and predicts content for a plurality of predicted user queries 412, 413 and a plurality of predicted responses 414, 415 in parallel based on the text input 207 and the conversation history 401. FIG. 4 depicts the plurality of predicted user queries as a predicted user query 412 and a predicted user query 413 for clarity. However, in various embodiments, the plurality of predicted user queries includes more than two predicted user queries. Also, FIG. 4 depicts the plurality of predicted responses as a predicted response 414 and a predicted response 415 for clarity. However, in various embodiments, the plurality of predicted responses includes more than two predicted responses.

To predict the plurality of predicted user queries 412, 413 and the plurality of predicted responses 414, 415, the text generator 408 extracts prior interactions from the conversation history 401 to identify a process of how future queries are provided based on a conversation and the responses to those questions using pattern recognition and a sequential LLM inference. Also, the text generator 408 identifies identities, context, and intent from the text input 207. To illustrate via an example, the text generator 408 extracts the text input 207 content as a question, “What are the best colleges in my state?” The text generator 408 extracts other types of questions and responses from conversation history as “What grades do I need for college?”, “What extracurricular activities help my college candidacy?”, “The best colleges often look for a grade point average over 3.5”, and “Participation on the debate team is favored by the best colleges”, respectively. The text generator 408 sends the plurality of predicted user queries 412, 413 to the embedding model 410 and the plurality of predicted responses 414, 415 to the RVG 418. The embedding model 410 converts each of the plurality of predicted user queries 412, 413 into a high dimensional vector as a plurality of embedded predicted user queries (EPUQ) 422, 423. The embedding model 410 embeds each of the plurality of predicted user queries 412, 413 into a context-aware vector that is stored in the embedded database within the storage unit 140. By employing a vector format to store the plurality of EPUQ 422, 423, the embedding model 410 generates an index within the embedded database that facilitates rapid retrieval and context awareness. Moreover, the text generator 408 anticipates follow-up questions to a given query to match responses to those queries for faster response times and substantially eliminate (e.g., nearly zero) latency.

The RVG 418 generates (i.e., precomputes) a plurality of predicted response variants (PRV) for each of the plurality of predicted responses 414, 415. Specifically, the RVG 418 generates a set of a plurality of PRV 424, 425 for the predicted response 414 and another set of a plurality of PRV 426, 427 for the predicted response 415. As such, the plurality of PRV 424, 425 represents alternative types of responses for answering a first query relevant to those responses. Similarly, the plurality of PRV 426, 427 represent alternative type of responses for answer a second query different from the first query. For example, a first query from the user “how can I improve my sleep?” The PRV 424 may be a response related to better sleep hygiene while the PRV 425 may be a response discussing cognitive behavioral therapy. As another example, a second query from the user “what can help me with meditation?” The PRV 426 may discuss aroma therapy while the PRV 427 may discuss using calm music. Accordingly, the RVG 418 diversifies the AI persona 102 response to prevent repetitive output. The RVG 418 performs multi-pass validation to ensure contextual accuracy for each predicted response. Also, the plurality of PRV 424, 425, 426, 427 are personalized responses based on interaction history with the user. The RVG 418 stores the precomputed queries in the embedded database. The RVG 418 stores the precomputed responses within the PR database. FIG. 4 depicts the plurality of PRV 424, 425 as the PRV 424 and the PRV 425 for clarity. However, in various embodiments, the plurality of PRV includes more than two PRVs. The same description applies to the plurality of PRV 426, 427.

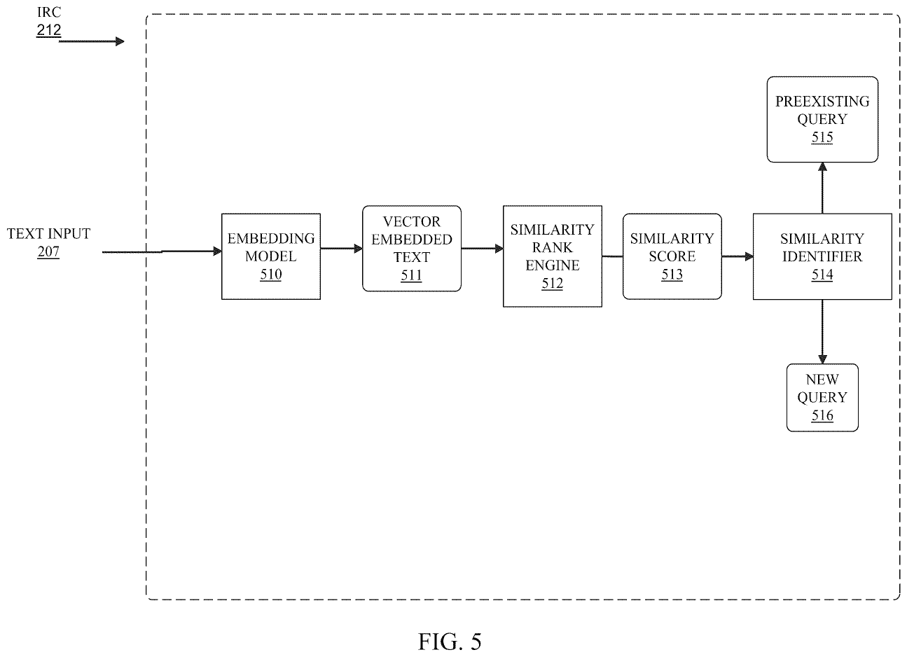

FIG. 5 is a block diagram of an input recognition and comparison (IRC) module 212 to compare a query to a preexisting stored query in a database using vector similarity matching and retrieve a precomputed response for a matched query in accordance with some embodiments. To ensure accuracy in response to queries, the AI persona 102 employs the IRC module 212 to evaluate whether the text input 207 is new query or is a preexisting query (i.e., same or similar to a prior query). In some embodiments, the IRC module 212 includes an embedding model 510, a similarity rank engine (SRE) 512, and a similarity identifier module 514, but is not limited thereto. The embedding model 510 converts the text input 207 into a high dimensional context-aware vector as a vector embedded text 511. The embedding model 510 employs deep learning embedding models to perform the conversion. The embedding model 510 sends the vector embedded text 511 to the SRE 512.

The SRE 512 compares the vector embedded text 511 to prior queries. In some embodiments, the SRE 512 may retrieve the prior queries (e.g., EPUQ 422, 423) from the embedded database, not shown for clarity. In some embodiments, the SRE 512 employs a cosine similarity algorithm to generate at least one similarity score 513 based on comparisons to each of the prior queries. That is, there may be a plurality of similarity scores 513 for the plurality of EPUQ 422, 423. The SRE 512 sends the at least one similarity score 513 to the similarity identifier 514. The similarity identifier 514 compares the at least one similarity score 513 to a similarity threshold. Thus, in some embodiments, the similarity identifier 514 indicates the vector embedded text 511 is similar to at least one prior query in response to the at least one similarity score 513 exceeding and/or matching (e.g., greater than or equal to) the similarity threshold. As such, the similarity identifier 514 indicates the vector embedded text 511 is not similar to at least one prior query in response to the at least one similarity score 513 falling below the similarity threshold. As described above, the vector embedded text 511 matching the similarity threshold indicates at least one precomputed response (e.g., the PRV 424, 425, 426, 427) may be used and can be rapidly extracted. In this manner, the precomputed or cached response substantially reduces latency and provides a faster response for engagement with the user. Alternatively, the vector embedded text 511 falling below the similarity threshold requires the TG module 216 to generate the generated response 217 to provide a response based on the new query.

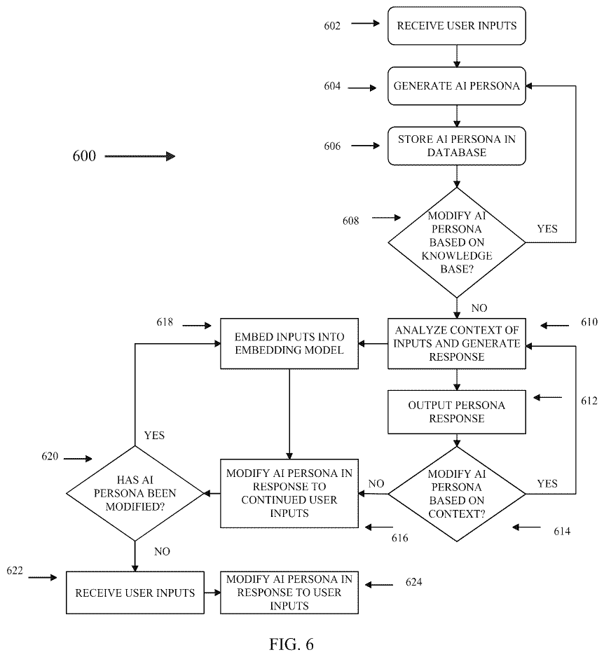

FIG. 6 is a flow diagram illustrating a method 600 for generating the AI persona 102 that changes based on a plurality of inputs in accordance with some embodiments. The method 600 is described with respect to an example implementation of the AI system 100 of FIG. 1 and FIG. 2. At block 602, the visual input device 160 detects a face input 103 in response to a trigger, such as movement detection within a predetermined distance and within a predetermined area. Alternatively, and/or in addition thereto, the visual input device 160 scans and detects the face input 103 in response to a command input received by the visual input device 160. Also, the audio input device 162 detects an audio input 104 in response to a trigger, such as movement detection within a predetermined distance and within a predetermined area. Alternatively, and/or in addition thereto, the audio input device 162 scans and detects the audio input 104 in response to a command input (e.g., record an utterance, record speech, etc.) received by the audio input device 162.

At block 604, based on instructions from the AI persona 102, the one or more processing units execute instructions to generate a persona. To exhibit natural reaction and human likeness, the AI persona 102 evaluates a plurality of inputs. For example, the AI persona 102 receives the face input 103 and the audio input 104 and generates outputs based on the face input 103 and the audio input 104. At block 606, in response to generating the persona, the AI persona 102 embeds content extracted from each data format into a vector that is stored in the embedded database within the storage unit 140.

At block 608, the AI persona 102 offers refinement of persona characteristics. The AI persona 102 will modify the visual output 105 and the audio output 106 as needed based on interaction with the user. Moreover, the AI persona 102 will modify conversation features of the visual output 105 and the audio output 106 via a feedback loop in response to detected changes in context from the face input 103 and/or the audio input 104. At block 610, the CEAAG module 210 retrieves context from the text query and detects emotional tone from the text input 207, the face input 103, and the audio input 104. The CEAAG module 210 examines spoken words, pitch, and tone from the audio input 104, as well as, facial movements and facial micro-expressions from the face input 103. In generating the audio output 106, the CEAAG module 210 combines inputs from the AI models, the deep learning models, and the facial recognition models to generate the audio output 106. At block 612, the CEAAG module 210 generates the audio output 106 with emotion based on the inputs. At block 614, the AI persona 102 checks whether the text input, user expressions, tone, and/or any other inputs have changed. If the inputs have changed, the method returns to block 610. If the inputs have not changed, the method moves on to block 616. At block 616, the AI persona 102 will further modify the response tone of the audio output 106 based on detected emotion from the inputs. The CEAAG module 210 modifies the audio output 106 employing speech synthesis to reflect the emotional state of the user including parameters, such as pitch, speed, cadence, and tone.

At block 618, in response to generating the persona, the AI persona 102 embeds content extracted from each data format into a vector that is stored in the embedded database within the storage unit 140. At block 620, the AI persona 102 checks whether the persona has been modified based on changes to the inputs. If the inputs have changed, the method returns to block 618. If the AI persona 102 moves on to block 622. At block 622, the AI persona 102 receives additional inputs during user interaction, such as the text input 207, the face input 103, and/or the audio input 104. At block 624, the AI persona 102 will modify conversation features of the visual output 105 and the audio output 106 via a feedback loop in response to detected changes in context from the text input 207, the face input 103, and/or the audio input 104.

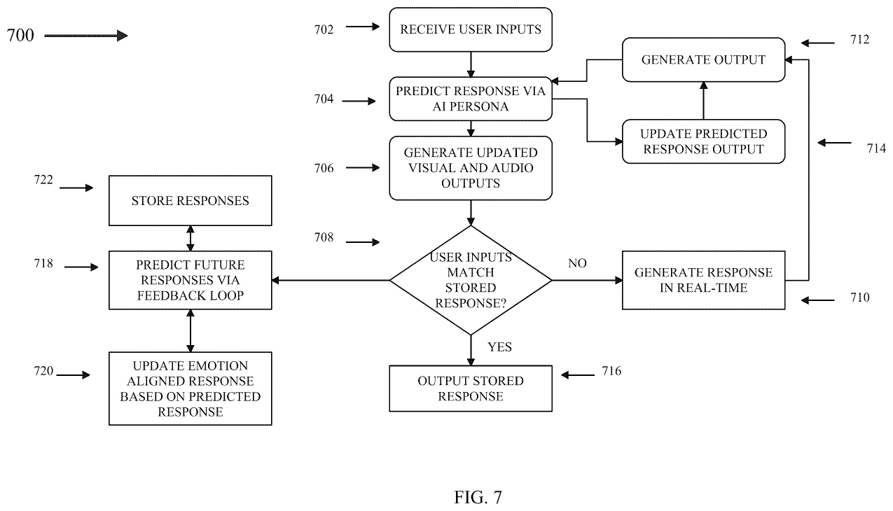

FIG. 7 is a flow diagram illustrating a method 700 for generating responses of an AI persona 102 based on matching a query and updating responses by learning facial expressions and emotions in accordance with some embodiments. The method 700 is described with respect to an example implementation of the AI system 100 of FIGS. 1-4. At block 702, the visual input device 160 detects a face input 103 in response to a trigger, such as movement detection within a predetermined distance and within a predetermined area. Alternatively, and/or in addition thereto, the visual input device 160 scans and detects the face input 103 in response to a command input received by the visual input device 160. Also, the audio input device 162 detects an audio input 104 in response to a trigger, such as movement detection within a predetermined distance and within a predetermined area. Alternatively, and/or in addition thereto, the audio input device 162 scans and detects the audio input 104 in response to a command input received by the audio input device 162.

At block 704, the text generator 408 receives and analyzes the text input 207 and a conversation history 401 (e.g., additional utterance and input or query from the user) to predict future queries and responses. The text generator 408 generates and predicts content for a plurality of predicted user queries 412, 413 and a plurality of predicted responses 414, 415 in parallel based on the text input 207 and the conversation history 401. At block 706, the AI persona 102 offers refinement of persona characteristics. The AI persona 102 will modify conversation features of the visual output 105 and the audio output 106 via a feedback loop in response to detected changes in context from the text input 207, the face input 103 and/or the audio input 104. At block 708, in response to receiving the text query, the AI persona 102 evaluates the text query using an IRC module. Specifically, the IRC module checks whether the text query has been input before, and therefore, is categorized as a preexisting query, or is instead a new query. At block 710, in response to the IRC detecting a new query, the TG module generates a new response in response to the IRC module confirming absence of the PQ within the embedded database. At block 712, the AI persona 102 presents at least one of the plurality of responses based on the new query, as well as additional context. At block 714, the AI persona 102 employs the CAE 214 to precompute queries and responses to quickly offer cached responses from further inputs and reduce latency. The CAE 214 stores the precomputed queries in the embedded database and the precomputed responses within the PR database. At block 716, for a preexisting query (PQ), the IRC module confirms presence of the PQ within an embedded database generated by the AI persona 102 and stored within the storage unit 140. A response lookup module retrieves a cached response from a preexisting response (PR) database generated by the AI persona 102 within the storage unit 140 for near-instantaneous delivery of a response.

At block 718, the AI persona 102 will modify conversation features of the visual output 105 and the audio output 106 via a feedback loop in response to detected changes in context from the text input 207, the face input 103 and/or the audio input 104. The feedback loop is real-time adaptation by the AI persona 102 to continuous input changes from the user and adjust the persona accordingly. At block 720, the modification of communication by the AI persona 102 may include extracting different predicted responses already stored in the storage unit 140 or generating a new response depending on the query input from the user. At block 722, the RVG 418 stores the precomputed responses within the PR database.

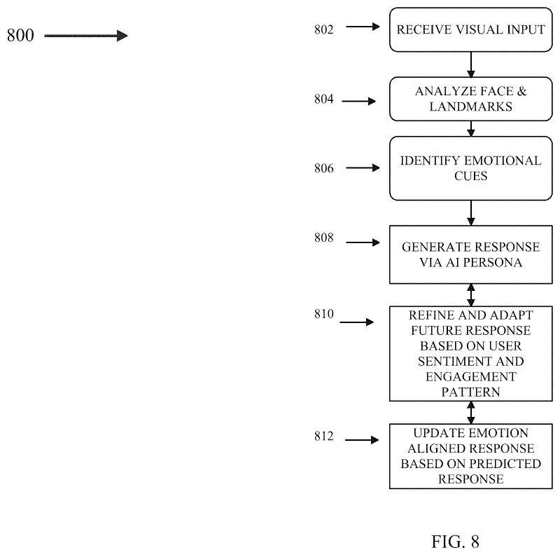

FIG. 8 is a flow diagram illustrating a method 800 for generating an AI persona 102 by analyzing facial expressions and identifying emotions in accordance with some embodiments. The method 800 is described with respect to an example implementation of the AI system 100 of FIGS. 1-4. At block 802, the visual input device 160 detects a face input 103 in response to a trigger, such as movement detection within a predetermined distance and within a predetermined area. Alternatively, and/or in addition thereto, the visual input device 160 scans and detects the face input 103 in response to a command input received by the visual input device 160. After the visual input device 160 detects and scans the face input 103, the visual input device 160 sends the face input 103 to the memory 103 via the I/O controller 170 for processing by the one or more processing units.

At block 804, the CEAAG module 210 employs facial recognition models. The CEAAG module 210 extracts facial expressions, facial micro-expressions, and eye gaze patterns. Also, the CEAAG module 210 analyzes facial landmarks for positioning and orientation, such as eye location, nose location, ear location, mouth location, and the like. At block 806, the CEAAG module 210 retrieves context from the text query and detects emotional tone from the text input 207, the face input 103, and the audio input 104. The CEAAG module 210 examines spoken words, pitch, and tone from the audio input 104, as well as, facial movements and facial micro-expressions from the face input 103. At block 808, the AI persona 102 employs the EAAPG module 222 to generate an animated model and outputs a visual output 105. The EAAPG module 222 synchronizes facial expressions, lip movements, and micro-expressions. The EAAPG module 222 responds in real-time to exhibit a natural and human-like persona. At block 810, the AI persona 102 offers refinement of persona characteristics. The AI persona 102 will modify the visual output 105 and the audio output 106 as needed based on interaction with the user. At block 812, the modification of communication by the AI persona 102 may include extracting different predicted responses already stored in the storage unit 140 or generating a new response depending on the query input from the user.

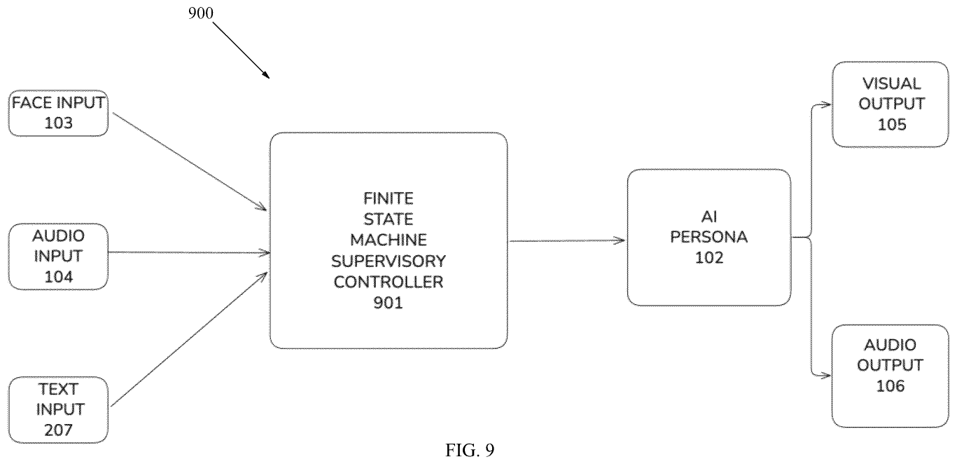

FIG. 9 is a block diagram of an AI system 900 that modifies inputs to an AI persona 102 in accordance with some embodiments. The Finite State Machine Supervisory Controller (FSMSC) 901 provides real-time safety monitoring and control for AI Persona 102 therapy sessions. The system analyzes patient emotional state through multimodal inputs and automatically restricts AI Persona 102 responses when detecting signs of distress or crisis. This controller 901 operates as a protective layer between the AI Persona 102 and patient, ensuring therapeutic interactions remain within safe parameters.

The FSMSC 901 integrates into the existing AI system as shown in FIG. 9. The controller 901 receives three input streams including the face input 103, the audio input 104, and the text input 207. In addition to obtaining the text input 207 as described above, in some embodiments, the text input 207 is a direct text input from an input device such as an input keyboard or a stylus, or may be text transcribed from speech using automatic speech recognition. When transcribed from speech, the system converts the patient's spoken words into written text.

The controller 901 processes these inputs to determine a safety state (NORMAL, CAUTION, or SAFE-HOLD) that governs the AI Persona's 102 response generation. Based on the current safety state, the controller 901 modulates the visual output 105 and the audio output 106. For the visual output 105, the controller 901 modulates the animated AI persona avatar, including facial expressions, lip movements, and micro-expressions, may be constrained to display calming, neutral expressions during elevated safety states, with the avatar presenting a supportive, non-reactive demeanor during SAFE-HOLD state. For example, a supportive, non-reactive demeanor comprises maintaining steady, calm visual presentation such as gentle eye contact, softened facial features, and reduced animation rate without mirroring or amplifying the patient's emotional distress. For the audio output 106, the controller 901 modulates speech synthesis to be limited or suspended entirely based on safety state, preventing the AI persona 102 from generating potentially harmful audio responses. For example, potentially harmful audio responses include probing questions that may escalate distress (for example, “Why do you feel that way?”), emotionally charged language, or directive statements that could be misinterpreted during crisis (for example, “You need to calm down”).

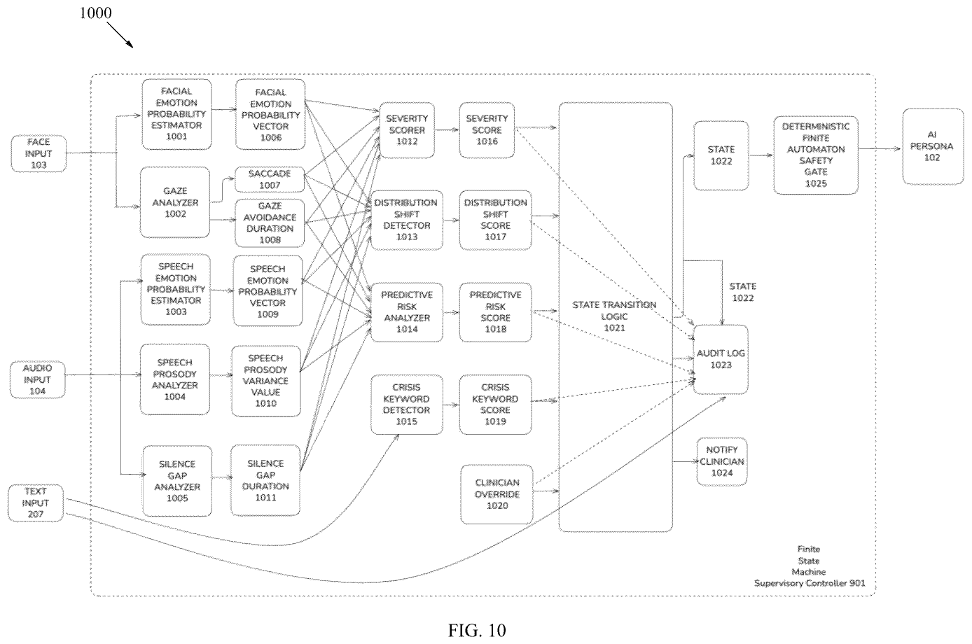

FIG. 10 is a block diagram of a finite state machine supervisory controller (FSMSC) 901 of an AI system in accordance with some embodiments. The FSMSC 901 comprises five functional stages: input processing, feature extraction, risk assessment, state management, and output control

### Face Input Processing Components (Blocks 1001-1002)

The system captures and analyzes multiple anatomical features from the patient's face such as: Eye Region Data (Pupil position coordinates relative to iris boundaries, Eyelid openness measurements, Eyebrow position and curvature, Presence or absence of crow's feet wrinkles near eye corners), Mouth Region Data (Lip corner positions (raised, neutral, or lowered), Mouth openness measurements, Presence or absence of smile lines, Upper lip curvature indicators), Nose and Cheek Region Data (Nasolabial fold depth (lines running from nose to mouth corners), Cheek elevation measurements, Nose wrinkle patterns), Overall Face Data (Forehead tension indicators, Face orientation in three-dimensional space, Head position and tilt angles).

The Facial Emotion Probability Estimator 1001 processes normalized face images through a trained neural network to generate probability distributions across defined emotion categories. The emotion categories may include the following (or more): happiness, sadness, anger, fear, surprise, disgust, and neutral. For each video frame, the facial emotion probability estimator 1001 generates a probability vector containing one value per emotion category. Each probability value ranges from zero to one, and the values within each frame's vector sum to one.

The Gaze Analyzer 1002 tracks pupil position relative to iris boundaries to compute three-dimensional gaze direction vectors. The gaze analyzer 1002 detects rapid eye movements (saccades) characterized by angular changes exceeding a defined value within defined time window in milliseconds. Sustained gaze avoidance is measured when the patient looks away from the screen (in other words, gaze direction vector angle exceeding a defined value) for periods exceeding a defined time interval in milliseconds.

### Audio Input Processing Components (Blocks 1003-1005)

The patient's speech audio is divided into short temporal segments. For each audio segment, the system extracts multiple acoustic features such as base pitch analysis, resonance frequency analysis, voice quality measures, speaking rate analysis, and mel-frequency cepstral coefficients (MFCCs).

For base pitch analysis, the system extracts the fundamental frequency (commonly called F0), which represents the base pitch of the patient's voice. This is typically computed using autocorrelation methods with time lag ranges or using specialized algorithms such as YIN, Probabilistic YIN (PYIN), cepstral peak detection, or sawtooth waveform inspired pitch estimator (SWIPE) spectral methods. For resonance frequency analysis, the system extracts formant frequencies (labeled F1, F2, F3, F4) which are the resonant frequencies that shape vowel sounds. Different emotional states cause subtle shifts in formant patterns due to changes in vocal tract configuration. For voice quality measures, the system measures micro-variations in pitch called jitter, micro-variations in loudness called shimmer, and the harmonics-to-noise ratio which indicates voice clarity versus breathiness. Emotional stress often increases jitter and shimmer while decreasing the harmonics-to-noise ratio.

For speaking rate analysis, the system computes the number of words or syllables spoken per minute. Anxiety and agitation often increase speaking rate, while sadness or depression may decrease it. For MFCCs, the system extracts MFCCs, which are numerical representations of the short-term power spectrum of the audio signal. MFCCs capture characteristics of the audio in a way that mimics human auditory perception, making them particularly useful for emotion recognition. In some embodiments, typically twelve to twenty MFCC coefficients are extracted per audio frame by the system.

In some embodiments, the Speech Emotion Probability Estimator 1003 analyzes acoustic features including fundamental frequency (F0), formant frequencies (F1-F4), jitter, shimmer, harmonics-to-noise ratio, and Mel-frequency cepstral coefficients. These features feed into a trained model to generate probability distributions across defined emotion categories. The emotion categories may include the following (or more): happiness, sadness, anger, fear, surprise, disgust, and neutral. Each probability ranges from zero to one with the sum equaling one. The estimator 1003 updates continuously for each audio segment.

In some embodiments, the Speech Prosody Analyzer 1004 measures pitch variation by computing variance in fundamental frequency over sliding temporal windows of defined length. The variance is normalized to a value between 0 and 1 using a saturating function with a clinically configured reference variance parameter to produce the speech prosody variance value 1010. In some embodiments, the Silence Gap Analyzer 1005 employs voice activity detection using frame energy thresholds and zero-crossing rates as described below. Silence gaps are identified as consecutive non-speech frames exceeding defined time windows. Gap durations in milliseconds are normalized to a value between 0 and 1 using a saturating function with a clinically configured reference duration parameter to produce the silence gap duration 1011.

The Silence Gap Analyzer 1005 distinguishes speech frames from silence frames using two acoustic measurements, frame energy and zero-crossing rate. For frame energy, the system calculates the total acoustic energy in the frame by summing the squared amplitude values of all audio samples in that frame, then converting to decibel scale using logarithmic transformation. Speech frames typically have higher energy compared to silence frames. For zero-crossing rate, the system counts how many times the audio waveform crosses the zero amplitude line within the frame, normalized by the total number of samples. Speech typically has zero-crossing rates between zero point one and zero point four, while pure noise or silence has different characteristic ranges.

The Facial Emotion Probability Vector 1006 contains probability values for each emotion category, updated at video frame rate by the facial emotion probability estimator 1001. The saccade 1007 has a normalized value between 0 and 1 computed as the ratio of frames containing saccadic eye movements to total frames within a sliding temporal window. A saccadic eye movement is detected when angular velocity exceeds a defined threshold. A saccade value of 0 indicates no rapid eye movements detected during the window period, while a value of 1 indicates continuous rapid eye movements throughout the window. The Gaze Avoidance Duration 1008 has a normalized value between 0 and 1 computed as the ratio of gaze-averted frames to total frames within a sliding temporal window. A frame is classified as gaze-averted when the gaze direction vector angle exceeds a defined threshold from screen-directed orientation. A value of 0 indicates the patient maintained screen-directed gaze throughout the window, while a value of 1 indicates complete gaze avoidance during the window period. The Speech Emotion Probability

Vector 1009 contains probability values for each emotion category, updated continuously during active speech by the speech emotion probability estimator 1003. The Speech Prosody Variance Value 1010 is computed as the variance of fundamental frequency values within a sliding temporal window, then normalized to a value between 0 and 1 using the formula: normalized_variance=variance/(variance+reference_variance), where reference_variance is a clinically configured parameter. A value of 0 indicates no pitch variation (monotone speech), while values approaching 1 indicate high pitch variability associated with emotional arousal. The Silence Gap Duration 1011 includes a duration of detected silence gaps measured in milliseconds, normalized to a value between 0 and 1 using the formula: normalized_duration=duration/(duration+reference_duration), where reference_duration is a clinically configured parameter representing a typical concerning pause length. A value of 0 indicates no pause detected, values around 0.5 indicate pauses at the reference duration, and values approaching 1 indicate very long pauses suggesting cognitive processing difficulty or dissociation.

The Severity Scorer 1012 computes a three-stage weighted combination to produce a final severity score 1016 ranging from 0 to 1. In the first stage, the system computes a facial emotion severity value as a weighted sum of the facial emotion probability vector 1006 elements, where each emotion category is multiplied by a clinician-configured distress coefficient (negative emotions such as sadness, anger, and fear receive higher weights than positive emotions). In the second stage, the system computes a speech emotion severity value as a weighted sum of the speech emotion probability vector 1009 elements using the same or similar distress coefficients. In the third stage, the system computes the final severity score 1016 as a weighted sum of the facial emotion severity, the speech emotion severity, the saccade value 1007, the gaze avoidance duration value 1008, the speech prosody variance value 1010, and the silence gap duration value 1011, where each component is multiplied by a clinician-configured weight. Importance weights derive from clinical research correlating each indicator with patient distress outcomes; reliability weights account for measurement consistency, with involuntary signals (such as speech prosody and silence gaps) weighted higher than signals susceptible to voluntary masking (such as facial expressions). The Distribution Shift Detector 1013 calculates Mahalanobis distance between the current feature vector and patient's baseline distribution. The current feature vector comprises the facial emotion probability vector 1006, the saccade value 1007, the gaze avoidance duration value 1008, the speech emotion probability vector 1009, the speech prosody variance value 1010, and the silence gap duration value 1011. Distance values exceeding defined thresholds trigger corresponding safety states, with separate thresholds configured for CAUTION and SAFE-HOLD transitions. The distribution shift detector 1013 outputs the distribution shift score 1017.

The Predictive Risk Analyzer 1014 monitors trajectories of all severity components over defined time windows to detect escalation patterns. The predictive risk analyzer 1014 tracks the facial emotion severity, speech emotion severity, saccade value 1007, gaze avoidance duration value 1008, speech prosody variance value 1010, and silence gap duration value 1011, computing rate of change for each. The predictive risk analyzer outputs a predictive risk score 1018 between 0 and 1, where higher values indicate greater confidence of impending state escalation based on the observed trajectory patterns and acceleration of the component values. The Crisis Keyword Detector 1015 scans text input 207 for crisis language organized in two tiers. Tier 1 keywords (“hurt myself”, “kill myself”, “suicide”, “not worth living”) trigger immediate SAFE-HOLD. Tier 2 keywords (“can't cope”, “falling apart”) trigger CAUTION state. Detection uses both exact phrase matching and semantic similarity assessment. The crisis keyword detector 1015 outputs crisis keyword score 1019.

For outputs, the Severity Score 1016 includes a continuous value from 0 to 1 updated continuously during active interaction by the severity scorer 1012. The Distribution Shift Score 1017 includes Mahalanobis distance value representing statistical deviation from baseline distribution, output by the distribution shift detector 1013. The Predictive Risk Score 1018 includes a continuous value from 0 to 1 representing confidence of impending state escalation based on trajectory analysis by the predictive risk analyzer 1014. The Crisis Keyword Score 1019 includes a binary flag for Tier 1 detection indicating immediate crisis language, or graduated score from 0 to 1 for Tier 2 keyword presence indicating moderate concern language, output by the crisis keyword detector 1015.

Referring to FIG. 10, the Crisis Keyword Score 1019 includes a binary flag for Tier 1 detection indicating immediate crisis language, or graduated score from 0 to 1 for Tier 2 keyword presence indicating moderate concern language, output by the crisis keyword detector 1015. The State Transition Logic 1021 implements trigger priority hierarchy, for example: (1) Clinician Override 1020, (2) Crisis Keyword Score 1019, (3) Predictive Risk Score 1018, (4) Distribution Shift Score 1017, (5) Severity Score 1016. (This priority hierarchy might be changed by clinician.) Applies escalation bias selecting more protective state when triggers conflict. Enforces hysteresis using different thresholds for escalation versus de-escalation, with configured gaps between thresholds to prevent state oscillation. The state transition logic 1021 determines the next state 1022. The State 1022 maintains current safety state (NORMAL, CAUTION, or SAFE-HOLD) with minimum persistence requirements. CAUTION state requires 5-15 seconds of sustained triggers before activation. SAFE-HOLD activates immediately for Tier 1 keywords or within 2-5 seconds for other triggers. The Audit Log 1023 records all state transitions from state 1022 with cryptographic integrity using Merkle tree structure. Each entry contains timestamp, trigger values, state changes, and SHA-256 hash linking to previous entry. Logs are immutable and tamper-evident for regulatory compliance. Lastly, the Notify Clinician 1024 is a multi-channel alert system using dashboard notifications, mobile messages, and pager integration. CAUTION state triggers generate non-urgent notifications with patient ID, timestamp, and trigger values. SAFE-HOLD state triggers generate urgent alerts with session links and crisis keyword excerpts.

A Deterministic Finite Automaton Safety Gate 1025 filters AI responses based on current state 1022. Specifically, states of the Deterministic Finite Automaton Safety Gate 1025 include (1) NORMAL state: no restrictions on AI Persona 102 output via visual output 105 and audio output 106, (2) CAUTION state: Limits responses to 1-3 sentences, blocks emotionally charged topics, increases validation phrases (“That sounds difficult”), and constrains vocabulary to common terms, and (3) SAFE-HOLD state: Blocks all conversational output, displays holding message (“Your care team has been notified and will join shortly”) via visual output 105, maintains video/audio capture for clinician review.

In normal state, the patient's emotional indicators remain within their personal baseline range. All triggers report values below CAUTION thresholds. The AI Persona 102 operates without restrictions, employing full therapeutic techniques. For example, a patient discusses work stress with severity score 1016 below CAUTION threshold and distribution shift score 1017 within normal range. The AI Persona 102 engages in cognitive behavioral exploration, asking open-ended questions about thought patterns. Session continues normally with periodic monitoring. In caution state, moderate distress indicators exceed baseline patterns. Severity score 1016 exceeds CAUTION threshold but remains below SAFE-HOLD threshold, or distribution shift score 1017 exceeds defined CAUTION threshold. The AI persona 102 operates under protective constraints while maintaining therapeutic engagement. For example, a patient becomes tearful discussing family conflict, severity score 1016 rises above CAUTION threshold, speech prosody variance value 1010 indicates elevated pitch variability. System enters CAUTION via state transition logic 1021, AI Persona 102 responses become brief and supportive: “This is really hard for you. Let's take a moment.” Clinician receives notification via Notify Clinician 1024 and monitors remotely. In safe-hold state, severe distress signals indicate crisis risk. Severity score 1016 exceeds SAFE-HOLD threshold, distribution shift score 1017 exceeds defined SAFE-HOLD threshold, or Tier 1 crisis keywords detected by crisis keyword detector 1015. AI Persona 102 conversation suspended immediately by deterministic finite automaton safety gate 1025. For example, if a patient types “I want to hurt myself”, triggering immediate SAFE-HOLD via state transition logic 1021. AI Persona 102 displays via visual output 105: “Your care team has been notified and will join shortly. You're not alone.” Clinician receives urgent alert via notify clinician 1024 and joins session within 90 seconds, finding patient in acute distress requiring direct intervention.

### Baseline Distribution Establishment

Initial calibration requires 3-5 sessions confirmed crisis-free by supervising clinician. During calibration, the system continuously collects the six feature extraction outputs 1006-1011: the facial emotion probability vector 1006, the saccade value 1007, the gaze avoidance duration value 1008, the speech emotion probability vector 1009, the speech prosody variance value 1010, and the silence gap duration value 1011. From this collected data, the system computes two baseline statistics: (1) the mean baseline feature vector, calculated as the average value of each of the six components across all calibration samples, representing the patient's typical emotional and behavioral profile; and (2) the covariance matrix, calculated from the variations in the six components across calibration samples, capturing how the components vary together and the typical range of variability for this patient. These baseline statistics are stored in the patient's profile and used by the distribution shift detector 1013 to compute Mahalanobis distance during subsequent sessions. Baseline updates incrementally at 1-5% learning rate per session, excluding crisis events to prevent contamination.

State 1022 transitions enforce temporal consistency: CAUTION entry: 5-15 seconds sustained triggers, SAFE-HOLD entry: Immediate for keywords, 2-5 seconds for other triggers, De-escalation: 10-30 seconds of improved indicators, and predictive risk assessment: based on 2-5 minute trajectory windows.

Default clinical thresholds (adjustable per patient): Severity: Separate thresholds configured for CAUTION and SAFE-HOLD transitions, Distribution: Separate thresholds configured for CAUTION and SAFE-HOLD transitions, Prosody variance: Threshold configured for significant deviation detection, Gaze avoidance: Threshold configured for concerning avoidance ratio detection, Silence gaps:Threshold configured for abnormal pause detection, and Predictive trajectory:Threshold configured for escalation warning detection.

Data preservation includes SAFE-HOLD state maintains: Rolling buffer of 2-5 minutes pre-trigger video/audio, Complete multimodal feature vectors at 50 Hz sampling, Full session transcript with timestamp synchronization, Cryptographic audit trail with SHA-256 integrity verification, and Automated backup to HIPAA-compliant cloud storage.

For authentication and authorization, Clinician override requires: Valid medical license verification, Role-based access control (attending/supervisor privileges), Two-factor authentication (password plus mobile token), Session-specific authorization code, and M-of-N acknowledgment for team-based decisions.

Patient privacy is preserved through on-device processing when possible, with only aggregate metrics transmitted to clinical dashboards. Raw video 103 and audio 104 remain encrypted at rest and in transit using AES-256 encryption. This safety control system provides comprehensive protection while preserving therapeutic benefit, balancing automated monitoring with human clinical judgment to ensure optimal patient outcomes.

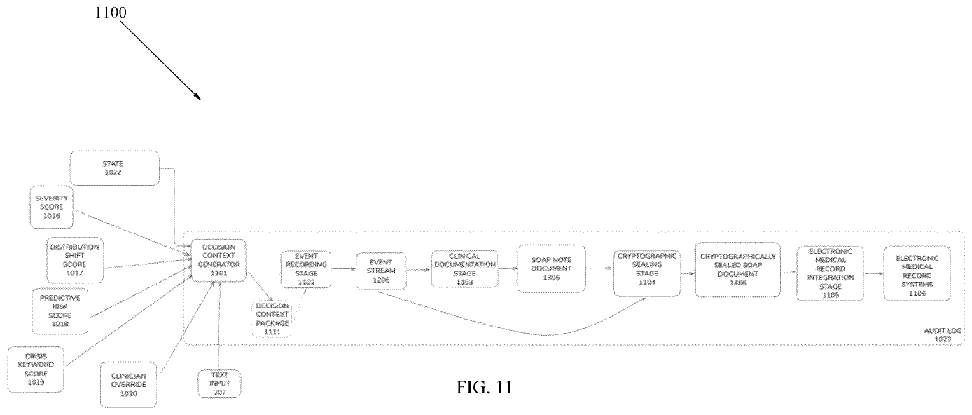

FIG. 11 is a block diagram of an audit log 1023 of the FSMSC 901 in accordance with some embodiments. The Audit Log 1023 provides comprehensive trust and auditability capabilities for AI-persona therapy sessions. These internal components capture safety events from State Transition Logic 1021, transform them into standard clinical documentation formats, apply cryptographic sealing for medicolegal integrity, and transmit records to hospital Electronic Medical Record (EMR) systems. This creates an unalterable audit trail that clinicians trust and regulatory bodies can verify, enabling safe deployment of AI therapy systems in clinical settings. FIG. 11 illustrates the internal architecture of Audit Log 1023. As shown in FIG. 11, Audit Log 1023 comprises Decision Context Generator 1101, which receives inputs from State Transition Logic 1021, and a processing pipeline that transforms safety events into cryptographically sealed clinical documentation for Electronic Medical Record Systems 1106.

Input to Audit Log 1023 via State Transition Logic 1021: State Transition Logic 1021 aggregates safety-relevant data from multiple sources and forwards comprehensive event packages to Audit Log 1023. This bridge architecture ensures that Audit Log 1023 receives complete context for each state transition, including (1) State 1022: Current safety state (NORMAL, CAUTION, SAFE-HOLD) and state transition events determined by State Transition Logic 1021, (2) Severity Score 1016: Continuous distress measurement that State Transition Logic 1021 evaluates for threshold crossings, (3) Distribution Shift Score 1017: Baseline deviation measurement that State Transition Logic 1021 monitors for anomalies, (4) Predictive Risk Score 1018: Escalation prediction that State Transition Logic 1021 considers for preemptive state changes, (5) Crisis Keyword Score 1019: Crisis language detection that State Transition Logic 1021 prioritizes for immediate response, (6) Clinician Override 1020: Manual interventions that State Transition Logic 1021 processes with highest priority, and text input 207.

State Transition Logic 1021 forwards these inputs to Decision Context Generator 1101, which enriches the raw data with contextual information. This ensures the audit trail captures not just what happened, but why the system made each decision. Decision Context Generator 1101 receives raw safety data from State Transition Logic 1021 and transforms it into structured decision context packages suitable for audit documentation. As shown in FIG. 11, Decision Context Generator 1101 sits between State Transition Logic 1021 and Event Recording Stage 1102, serving as the bridge that enriches operational data with audit-ready context. Decision Context Generator 1101 receives: State 1022 values, Severity Score 1016, Distribution Shift Score 1017, Predictive Risk Score 1018, Crisis Keyword Score 1019, Clinician Override 1020, and Text Input 207. For each state transition event, Decision Context Generator 1101 produces a decision context package that documents the complete reasoning behind each state determination. The decision context package comprises six elements including (1) Trigger Attribution: Identifies which specific input triggered the state change. The trigger is one of: Severity Score 1016 exceeding threshold, Distribution Shift Score 1017 exceeding threshold, Predictive Risk Score 1018 indicating imminent escalation, Crisis Keyword Score 1019 detecting crisis language, or Clinician Override 1020 manually setting state. The trigger attribution records which input was the primary cause of the state transition; (2) Priority Evaluation Results: Documents how State Transition Logic 1021 applied the priority hierarchy when multiple triggers exceeded thresholds simultaneously. State Transition Logic 1021 evaluates triggers in priority order: (1) Clinician Override 1020 takes highest priority, (2) Crisis Keyword Score 1019 takes second priority, (3) Predictive Risk Score 1018 takes third priority, (4) Distribution Shift Score 1017 takes fourth priority, (5) Severity Score 1016 takes fifth priority. (This priority hierarchy might be changed by clinician.) The priority evaluation results record which triggers were active and which trigger took precedence; (3) Threshold Comparison Values: Records the configured threshold value for each score and whether the current value exceeded, met, or fell below that threshold at the moment of evaluation. For Severity Score 1016, this includes both the CAUTION threshold and the SAFE-HOLD threshold. For Distribution Shift Score 1017, this includes the Mahalanobis distance thresholds for CAUTION and SAFE-HOLD transitions. The comparison documents the margin by which thresholds were exceeded or not met; (4) Hysteresis State: Indicates whether the transition was an escalation (NORMAL to CAUTION, or CAUTION to SAFE-HOLD) or a de-escalation (SAFE-HOLD to CAUTION, or CAUTION to NORMAL). For de-escalation transitions, State Transition Logic 1021 applies a hysteresis gap requiring scores to fall further below the threshold than required for escalation, preventing state oscillation. Decision Context Generator 1101 records the gap value applied and whether hysteresis requirements were satisfied; (5) Score Snapshot: Decision Context Generator 1101 captures all score values at the exact moment State Transition Logic 1021 made its decision: Severity Score 1016 value, Distribution Shift Score 1017 value (Mahalanobis distance), Predictive Risk Score 1018 value, and Crisis Keyword Score 1019 value. This snapshot enables post-hoc analysis of system behavior and provides evidence that the system responded appropriately to the measured conditions; and (6) State Transition Record: Decision Context Generator 1101 documents the previous State 1022 value (NORMAL, CAUTION, or SAFE-HOLD), the new State 1022 value, and the precise timestamp of transition with microsecond precision. For transitions triggered by Clinician Override 1020, this also includes the clinician identifier and justification text.

The Decision Context Generator 1101 outputs the complete decision context package 1111 to Event Recording Stage 1102, which serves as the entry point to Audit Log 1023. Next, are Internal Processing Stages within Audit Log 1023: (1) Event Recording Stage 1102: Captures state transitions, risk scores, and clinical actions into structured audit records with cryptographic hash chains, (2) Clinical Documentation Stage 1103: Transforms technical AI safety data into standard clinical SOAP (Subjective-Objective-Assessment-Plan) note format that healthcare providers understand, (3) Cryptographic Sealing Stage 1104: Applies tamper-evident cryptographic methods including Merkle trees, digital signatures, and trusted timestamps to ensure records cannot be altered after creation, and (4) EMR Integration Stage 1105: Encodes clinical documentation in HL7 FHIR (Fast Healthcare Interoperability Resources) format and transmits to hospital systems.

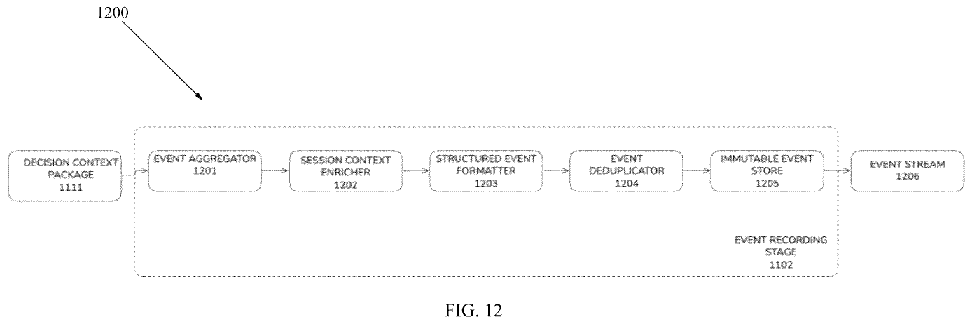

FIG. 12 is a block diagram of an event recording stage 1102 in accordance with some embodiments. Event Aggregator 1201: Receives decision context packages from Decision Context Generator 1101. Each decision context package contains the complete audit-ready information: the six context elements (trigger attribution identifying which input caused the state change, priority evaluation results showing how conflicting triggers were resolved, threshold comparison values documenting the margin of threshold exceedance, hysteresis state indicating escalation versus de-escalation, score snapshot capturing all values at decision time, and state transition record documenting the state change with timestamp), plus Text Input 207 transcripts for subjective documentation. The aggregator buffers these packages in a time-ordered queue with microsecond timestamp precision, preserving the complete decision context for downstream processing. Session Context Enricher 1202: Augments raw events with contextual metadata required for clinical interpretation. For each event received from Event Aggregator 1201, the enricher adds: patient demographic identifiers (medical record number, age, gender), session metadata (session ID, start time, duration, therapy protocol), clinician identifiers (attending physician, supervising therapist, care team members), and environmental context (location, device type, network quality indicators). Output produces enriched event records containing both technical data and clinical context.

Structured Event Formatter 1203: Transforms enriched events into standardized JSON schema optimized for clinical workflows. Each event from Session Context Enricher 1202 is structured with: primary event type (state_transition, risk_alert, clinician_action), timestamp with timezone information, triggering conditions (which thresholds were exceeded), multimodal indicator values at event time (facial emotion probabilities, gaze metrics, speech prosody values), applied safety interventions (conversation restrictions, topic blocks, response limits), and clinical significance flags (routine, concerning, critical). The formatter ensures consistent structure across all event types. Event Deduplicator 1204: Removes redundant events caused by system redundancy or network retransmissions. Computes hash of event content excluding timestamp, compares against recent event cache spanning previous 30 seconds, and suppresses duplicate events while preserving the earliest timestamp. This prevents audit log inflation while maintaining temporal accuracy. Immutable Event Store 1205: Persists deduplicated events in write-once storage with cryptographic integrity verification. Each event from Event Deduplicator 1204 is assigned a monotonically increasing sequence number, stored with SHA-256 content hash, linked to previous event hash creating hash chain, and written to append-only database preventing modification or deletion. The immutable store outputs Event Stream 1206 for downstream processing within Audit Log 1023.

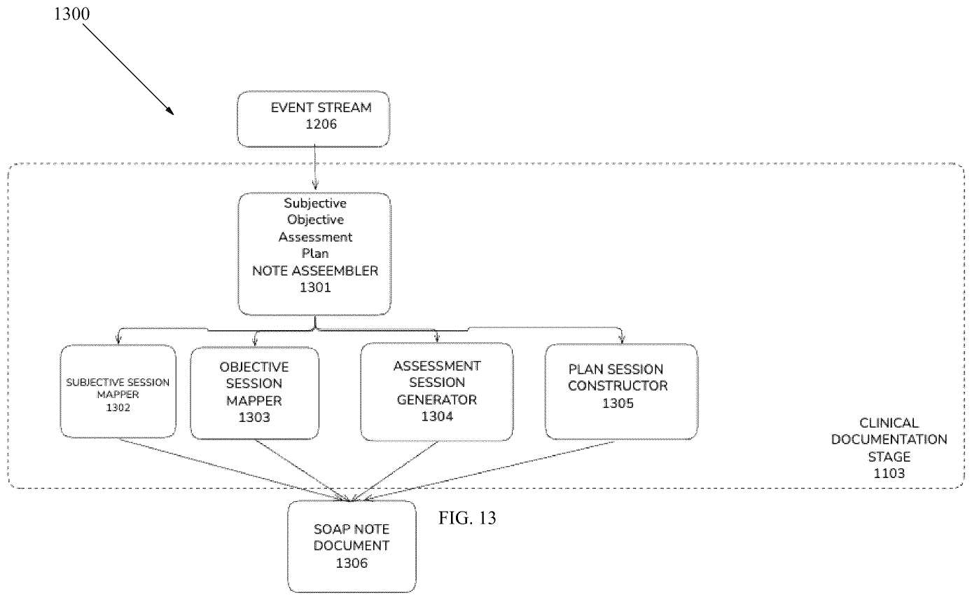

FIG. 13 is a block diagram of a clinical documentation stage 1103 in accordance with some embodiments. SOAP Note Assembler 1301: Orchestrates the transformation of technical event data into clinical documentation format. Receives Event Stream 1206 from Immutable Event Store 1205, identifies clinically significant patterns requiring documentation, and triggers specialized mappers for each SOAP section. The assembler maintains session state to ensure temporal consistency across SOAP sections. Subjective Section Mapper 1302: Extracts and formats patient-reported information for the Subjective section of the SOAP note. Input: Text Input 207 transcripts and crisis keywords from session events. Processing: Identifies chief complaints from initial patient statements, extracts symptom descriptions using natural language processing, highlights emotional expressions and concerns, and preserves exact patient phrases when clinically relevant. Output: Formatted text block containing patient's subjective experience, for example: “Patient reports feeling ‘overwhelmed by work stress’ and states ‘I don't know if I can handle this anymore.’ Denies suicidal ideation but expresses hopelessness about situation improving.”

Objective Section Mapper 1303: Transforms multimodal AI measurements into clinical observations for the Objective section. Input: Facial emotion probability vectors from Facial Emotion Probability Vector 1006, gaze metrics from Gaze Avoidance Duration 1008, speech prosody measurements from Speech Prosody Variance Value 1010, and behavioral indicators captured in event stream. Processing: Converts probability values to clinical descriptors (sadness probability 0.72 becomes “Patient exhibits marked facial sadness (72%)”), aggregates related metrics into clinical observations (combining gaze avoidance and saccade values into “Poor eye contact with frequent gaze aversion noted”), and translates technical measurements to medical terminology (speech prosody variance becomes “Voice demonstrates emotional lability with pitch variations”). Output: Structured clinical observations resembling traditional mental status exam findings. Assessment Section Generator 1304: Synthesizes safety state determinations and risk assessments into clinical interpretations. Input: Current safety State 1022 (NORMAL, CAUTION, SAFE-HOLD), triggering thresholds and scores from Severity Score 1016, Distribution Shift Score 1017, and Predictive Risk Score 1018, plus state transition history during session. Processing: Maps FSM states to clinical risk levels (SAFE-HOLD becomes “Acute distress requiring immediate intervention”), correlates multiple risk indicators into unified assessment, generates differential considerations based on symptom patterns, and assigns provisional diagnostic impressions when warranted. Output: Clinical assessment paragraph, for example: “Patient presenting with moderate emotional dysregulation (Safety State: CAUTION triggered at 14:23:45). Multimodal indicators suggest anxiety with depressive features. Risk assessment: Moderate-protective monitoring indicated but not acute crisis.”

Plan Section Constructor 1305: Documents applied interventions and recommended follow-up based on safety system actions. Input: Applied output constraints from Deterministic Finite Automaton Safety Gate 1025, clinician notifications from Notify Clinician 1024, manual overrides from Clinician Override 1020. Processing: Lists AI conversation restrictions as therapeutic boundaries (“Automated redirection from trauma processing implemented”), documents clinician alerts as care coordination (“Supervising therapist notified via secure message at 14:24:00”), includes recommended monitoring frequency based on risk level, and suggests clinical follow-up actions. Output: Structured plan documentation integrating automated and human-directed interventions. SOAP Note Document Assembly: SOAP Note Assembler 1301 aggregates the outputs from each specialized component to construct the complete SOAP note document. The Subjective section output from Subjective Section Mapper 1302 is placed under the “S” heading, the Objective section output from Objective Section Mapper 1303 is placed under the “O” heading, the Assessment section output from Assessment Section

Generator 1304 is placed under the “A” heading, and the Plan section output from Plan Section Constructor 1305 is placed under the “P” heading. SOAP Note Assembler 1301 ensures temporal consistency across sections, applies consistent formatting, and adds document metadata including session identifier, patient identifier, date, and time. The assembled SOAP note document 1306 is then forwarded to Cryptographic Sealing Stage 1104 for tamper-evident protection.

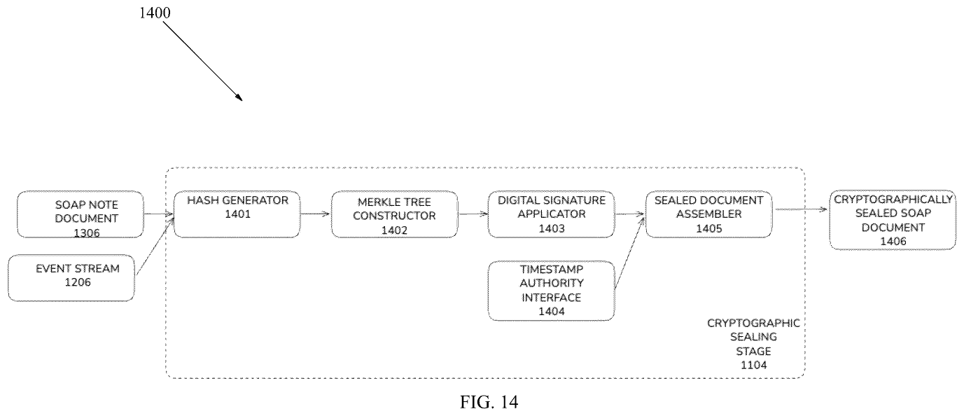

FIG. 14 is a block diagram of a cryptographic sealing stage 1104 in accordance with some embodiments. Hash Generator 1401: Creates cryptographic fingerprints for each documented element ensuring tamper detection. Input: Individual SOAP note sections from mappers 1302-1305 and event records from Event Stream 1206. Processing: Applies SHA-256 hashing algorithm to content bytes, generating 256-bit hash values unique to content. Even single character modifications produce completely different hash values. Each SOAP note section receives independent hash allowing granular integrity verification. Merkle Tree Constructor 1402: Builds hierarchical hash structure enabling efficient verification of large audit logs. Input: Sequential event hashes from Hash Generator 1401 for entire session. Processing: Organizes hashes into binary tree structure where leaf nodes contain individual event hashes, internal nodes contain hashes of child node pairs (Hash (left_child∥right_child)), and root node contains single hash representing entire session. This structure allows verification of any single event without accessing all events, while modification of any event invalidates the root hash.

Digital Signature Applicator 1403: Applies cryptographic signatures binding clinical responsibility to documentation. Input: Merkle tree root hash from Merkle Tree Constructor 1402 and clinician's private key. Processing: Generates digital signature using RSA-2048 or ECDSA algorithms, creates signature block containing: signer identification (name, medical license, NPI number), timestamp from trusted time source, signature algorithm identifier, and signature bytes. The signature mathematically proves the signing clinician reviewed and approved the documented session at the specified time. Timestamp Authority Interface 1404: Obtains cryptographically verifiable timestamps from trusted external source. Connects to RFC 3161-compliant timestamp server, submits hash of content requiring timestamp, receives timestamp token containing: certified time from atomic clock source, timestamp authority digital signature, certificate chain to trusted root. This provides legally admissible proof of document existence at specific time, preventing backdating or temporal manipulation. Sealed Document Assembler 1405: Combines clinical content with cryptographic proofs into tamper-evident package. Input: SOAP note content from SOAP Note Assembler 1301, Merkle tree structure from Merkle Tree Constructor 1402, digital signatures from Digital Signature Applicator 1403, timestamp tokens from Timestamp Authority Interface 1404. Processing: Creates sealed document containing: plain text SOAP note for clinical reading, cryptographic proof bundle (hashes, signatures, timestamps), verification instructions for integrity checking, and audit trail linking to source events. Output: Cryptographically sealed SOAP document 1406 ready for permanent storage and EMR transmission.

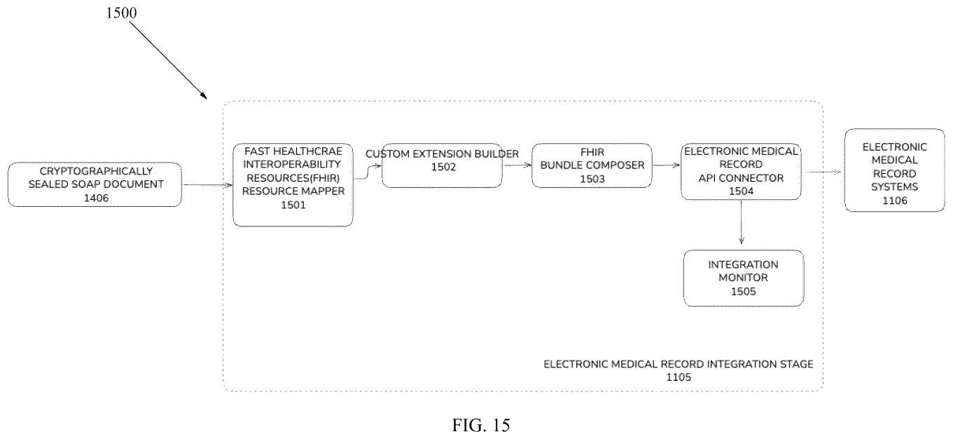

FIG. 15 is a block diagram of an electronic medical record (EMR) integration stage 1105 in accordance with some embodiments. FHIR Resource Mapper 1501: Transforms sealed clinical documentation into HL7 FHIR standard resources. Input: Sealed SOAP note and cryptographic proofs from Sealed Document Assembler 1405. Processing: Creates base FHIR Observation resource with required fields (resourceType, status, code, subject, effectiveDateTime), maps SOAP sections to FHIR narrative structure, and assigns appropriate LOINC and SNOMED-CT codes for clinical concepts. Each safety state maps to specific FHIR CodeableConcept enabling standardized queries. Custom Extension Builder 1502: Constructs FHIR extensions for AI-specific data not covered by standard FHIR. Creates extension definitions for: multimodal emotion indicators (facial and speech probabilities as structured data), FSM safety states (NORMAL, CAUTION, SAFE-HOLD as coded values), applied AI constraints (topic restrictions, response limits as operational data), and cryptographic provenance (Merkle root hash, digital signatures as security metadata). Extensions follow FHIR profiling guidelines ensuring compatibility with EMR systems while preserving AI-specific information.

FHIR Bundle Composer 1503: Packages related FHIR resources into transaction bundles for atomic EMR updates. Input: Multiple FHIR resources from session (observations, clinical notes, risk assessments). Processing: Creates FHIR Bundle resource with transaction type, assigns logical relationships between resources (SOAP note references triggering observations), includes provenance resources tracking data lineage, and sets transaction semantics (all-or-nothing commitment). Bundle ensures entire session documentation commits atomically or fails completely, preventing partial updates. EMR API Connector 1504: Manages secure transmission to hospital EMR systems with reliability guarantees. Establishes OAuth 2.0 authenticated connection to EMR FHIR endpoint, validates target EMR capabilities using FHIR CapabilityStatement, transmits FHIR Bundle using HTTPS with TLS 1.3 encryption, handles response codes and retry logic for transient failures, and logs transmission confirmations with EMR-assigned resource IDs. Supports major EMR platforms including Epic, Cerner, and Allscripts through standard FHIR interfaces. Integration Monitor 1505: Tracks documentation flow ensuring reliable delivery and compliance. Monitors successful transmissions versus failures, measures latency from event to EMR storage, detects missing or delayed documentation, generates compliance reports for regulatory audits, and triggers alerts for integration failures requiring manual intervention. Maintains metrics proving system meets documentation timeliness requirements.

As mentioned above, other embodiments and configurations may be devised without departing from the spirit of the invention and the scope of the appended claims.

1. A computer-implemented method for supervisory safety control during artificial intelligence clinical interaction, the method comprising:
maintaining a finite state machine supervisory controller configured to transition between a plurality of operational states based on patient risk indicators, the plurality of operational states comprising a normal state, a caution state, and a safe-hold state;
acquiring multimodal patient data during a clinical session, the multimodal patient data comprising facial emotional indicators derived from a face input, audio prosody indicators derived from an audio input, and linguistic content derived from a text input;
computing a severity score based on weighted analysis of the multimodal patient data, wherein the weighted analysis comprises applying clinician-configured distress coefficients to emotion probability values;
computing a distribution shift score by calculating a Mahalanobis distance between a current feature vector and an established patient baseline distribution;
automatically transitioning the finite state machine supervisory controller to an elevated safety state in response to at least one of the severity score exceeding a first configured threshold, the distribution shift score exceeding a second configured threshold, a predictive risk score indicating imminent escalation, and detection of crisis keywords in the linguistic content;
recording state transition events in a cryptographically secured audit log comprising a Merkle tree structure with hash chain integrity;
constraining artificial intelligence output generation based on the current operational state of the finite state machine supervisory controller, wherein the safe-hold state blocks conversational output and displays a holding message;
initiating authenticated notification to supervising clinical personnel upon entry to the safe-hold state; and
generating clinical documentation in SOAP format with cryptographic sealing for transmission to a downstream clinical system.

2. The method of claim 1, wherein the plurality of operational states comprises at least a normal state, a caution state, and a safe-hold state, and wherein transitioning between states includes hysteresis to prevent rapid oscillation.

3. The method of claim 1, wherein computing the risk assessment metric comprises:
weighted fusion of the visual emotional indicators, audio prosody indicators, and linguistic content; and
comparing current indicators against the established patient baseline using a distribution shift metric.

4. The method of claim 3, wherein the cryptographically secured audit trail comprises a Merkle tree structure with digitally signed root hashes.

5. The method of claim 1, wherein the finite state machine supervisory controller includes a clinician override interface requiring two-factor authentication and M-of-N acknowledgment protocol team-based override decisions.

6. The method of claim 1, wherein the clinical documentation comprises SOAP format notes with section-level cryptographic hashes generated by applying cryptographic digests and hash integrity to each SOAP section.

7. The method of claim 1, wherein transmitting to healthcare information systems comprises encoding the clinical documentation as FHIR resources and transmitting via HL7 FHIR API.

8. The method of claim 1, wherein the downstream clinical system comprises an Electronic Medical Record (EMR) system or an Electronic Health Record (EHR) system.

9. The method of claim 8, wherein transmitting to the EMR system or EHR system comprises:
encoding the clinical documentation as FHIR resources comprising at least an Observation resource encoding safety state transitions, and a Provenance resource with a digital signature of the Merkle tree root hash; and
transmitting the FHIR resources via HL7 FHIR API using HTTPS with authentication.

10. The method of claim 9, wherein the Provenance resource comprises:
a digital signature generated using encryption algorithms;
a timestamp from a trusted timestamp authority; and
a reference linking to the clinical documentation resources.

11. (canceled)

12. A system for supervisory safety control during artificial intelligence clinical interaction, the system comprising: one or more processing units, the one or processing units configured to:
maintain a finite state machine supervisory controller configured to transition between a plurality of operational states based on patient risk indicators, the plurality of operational states comprising a normal state, a caution state, and a safe-hold state;
acquire multimodal patient data during a clinical session, the multimodal patient data comprising facial emotional indicators derived from a face input, audio prosody indicators derived from an audio input, and linguistic content derived from a text input;
compute a severity score based on weighted analysis of the multimodal patient data, wherein the weighted analysis comprises applying clinician-configured distress coefficients to emotion probability values;
compute a distribution shift score by calculating a Mahalanobis distance between a current feature vector and an established patient baseline distribution;
automatically transition the finite state machine supervisory controller to an elevated safety state in response to at least one of the severity score exceeding a first configured threshold, the distribution shift score exceeding a second configured threshold, a predictive risk score indicating imminent escalation, and detection of crisis keywords in the linguistic content;
record state transition events in a cryptographically secured audit log comprising a Merkle tree structure with hash chain integrity;
constrain artificial intelligence output generation based on the current operational state of the finite state machine supervisory controller, wherein the safe-hold state blocks conversational output and displays a holding message;
initiate authenticated notification to supervising clinical personnel upon entry to the safe-hold state; and
generate clinical documentation in SOAP format with cryptographic sealing for transmission to a downstream clinical system.

13. The system of claim 12, wherein the downstream clinical system comprises an Electronic Medical Record (EMR) system or an Electronic Health Record (EHR) system, and wherein the processing units are configured to transmit clinical documentation to the EMR system or EHR system via HL7 FHIR API.

14. The system of claim 13, wherein the one or more processing units are further configured to:
encode clinical documentation as an FHIR Bundle comprising Observation and Provenance resources; and
transmit the FHIR Bundle via HTTPS with authentication to an EMR or EHR FHIR endpoint.

15. (canceled)

16. The system of claim 12, wherein the system implements security controls comprising:
symmetric encryption algorithms at rest for persistent storage including the audit log, clinical notes, and patient baseline statistics;
role-based access control with least-privilege assignments for workforce access;
authentication for API access; and
audit controls including a Merkle tree audit log with the Merkle tree root signed using encryption algorithms.

17. A computer-implemented method for generating cryptographically sealed clinical documentation for integration with downstream clinical systems, the method comprising:
generating a SOAP clinical note comprising Subjective, Objective, Assessment, and Plan sections based on clinical session data;
creating section-level cryptographic hashes by applying cryptographic digests and hash integrity to each SOAP section content;
constructing a Merkle tree audit log with leaf nodes representing state transition events and SOAP section hashes, with parent nodes computed recursively and a digitally signed root hash using encryption algorithms;
constructing a sealed export bundle comprising the SOAP note, the signed Merkle root, and state transition events; and
transmitting the sealed export bundle to a downstream clinical system.

18. The method of claim 17, wherein the downstream clinical system comprises:
at least one of an Electronic Medical Record (EMR) system and an Electronic Health Record (EHR) system; and
transmitting the sealed export bundle comprises:
encoding the SOAP note as an FHIR resource;
encoding state transition events as FHIR Observation resources;
encoding audit trail information as an FHIR Provenance resource with digital signature of the Merkle tree root hash; and
transmitting an FHIR Bundle comprising the resources to the EMR or EHR system via FHIR API with authentication.

19. The method of claim 18, wherein each section-level cryptographic hash is produced by applying cryptographic digests and hash integrity to a serialized representation of the section content combined with multimodal indicator values comprising facial emotion indicators, prosody variance, silence gap duration, gaze avoidance duration, and a timestamp.

20. (canceled)

21. (canceled)
